(19) United States
(12) Patent Application Publication
(10) Pub. No.: US 2002/0151494 A1
(43) Pub. Date: Oct. 17, 2002

(54) Novel amino acid sequences for human semaphorin-like polypeptides

(75) Inventors: Richard A. Shimkets, Guilford, CT (US); Elma Fernandes, Branford, CT (US); Corine Vernet, Branford, CT (US); Meijia Yang, East Lyme, CT (US); Ferenc L. Boldog, North Haven, CT (US); John L. Herrmann, Gilford, CT (US)

Correspondence Address: Ivor R. Elrifi, MINTZ, LEVIN, COHN, FERRIS,, GLOVSKY AND POPEO, P.C., One Financial Center, Boston, MA, 02111, US

(73) Assignee: CuraGen Corporation, New Haven, CT (US)

(21) Appl. No.: 10/003,152

(22) Filed: Nov. 2, 2001

Related U.S. Application Data
(63) Continuation of application No. 09/604,286, filed on Jun. 22, 2000.
(60) Provisional application No. 60/154,520, filed on Sep. 16, 1999.
(60) Provisional application No. 60/144,722, filed on Jul. 20, 1999.
(60) Provisional application No. 60/140,584, filed on Jun. 23, 1999.

(51) Int. Cl. A61K 38/17; C12Q 1/68; G01N 33/53; C07H 21/04; C07K 14/435; C12P 21/02; C12N 5/06

(52) U.S. Cl. 514/12; 530/350; 536/23.5; 435/7.1; 435/69.1; 435/325; 435/320.1; 435/6

(57) ABSTRACT

This application is drawn to novel amino acid sequences for mammalian polypeptides that have sequence similarity of human and mus musculus Semaphorin. The encoded novel polypeptides comprise about 590 amino acids.

### RELATED APPLICATIONS

This application claims priority to U.S. Ser. No. 09/604,286 filed Jun. 22, 2000, pending, which claims the benefit of U.S. Ser. No. 60/154,520 filed Sep. 16, 1999, abandoned; U.S. Ser. No. 60/144,722 filed Jul. 20, 1999, abandoned; and U.S. Ser. No. 60/140,584 filed Jun. 23, 1999, abandoned, all of which are incorporated herein by reference in their entirety.

### FIELD OF THE INVENTION

The invention relates to polynucleotides and the polypeptides encoded thereby.

### BACKGROUND OF THE INVENTION

Many biologically important proteins are secreted from the cell after crossing multiple membrane-bound organelles. These proteins can often be identified by the presence of sequence motifs referred to as “sorting signals” in the protein, or in a precursor form of the protein. These sorting signals can aid in targeting the proteins to their appropriate destination.

One type of sorting signal is a signal sequence, which is also referred to as a signal peptide or leader sequence. This signal sequence, which can be present as an amino-terminal extension on a newly synthesized polypeptide. A signal sequence possesses the ability to “target” proteins to an organelle known as the endoplasmic reticulum (ER).

The signal sequence takes part in an array of protein-protein and protein-lipid interactions that result in the translocation of a signal sequence-containing polypeptide through a channel within the ER. Following translocation, a membrane-bound enzyme, designated signal peptidase, liberates the mature protein from the signal sequence.

Secreted and membrane-bound proteins are involved in many biologically diverse activities. Examples of known, secreted proteins include, e.g., insulin, interferon, interleukin, transforming growth factor-beta, human growth hormone, erythropoietin, and lymphokine. Only a limited number of genes encoding human membrane-bound and secreted proteins have been identified thus far.

### SUMMARY OF THE INVENTION

The invention is based in part upon the discovery of novel nucleic acids and secreted polypeptides encoded thereby. Novel nucleic acids and polypeptides include SEC1, SEC2, SEC3, SEC4, SEC5, SEC6, SEC7, SEC8, SEC9, SEC10, SEC11, and SEC12 nucleic acids and polypeptides. These nucleic acids and polypeptides are collectively referred to herein as “SECX”.

Accordingly, in one aspect, the invention provides an isolated nucleic acid molecule that includes a SECX nucleic acid, e.g. any of SEQ ID NOs:1, 3, 5, 7, 9, 11, 13, 15, 17, 19, 21, 23, and 25. In some embodiments, the SECX nucleic acid encodes a SECX polypeptide, e.g., a polypeptide including the amino acid sequence of any of SEQ ID NOs:2, 4, 6, 8, 10, 12, 14, 16, 18, 20, 22, or 24, or a fragment, homolog, analog or derivative thereof. A nucleic acid can include, e.g., a nucleic acid sequence encoding a polypeptide at least 85% identical to a polypeptide comprising the amino acid sequences of a SECX polypeptide. The nucleic acid can be, e.g., a genomic DNA fragment, a cDNA molecule, or the like.

Also included within the scope of the invention is a vector containing one or more of the nucleic acids described herein, and a cell containing the vectors or nucleic acids described herein.

The invention is also directed to host cells transformed with a vector comprising any of the nucleic acid molecules described above.

In another aspect, the invention includes a pharmaceutical composition that includes a SECX nucleic acid and a pharmaceutically acceptable carrier or diluent.

In a further aspect, the invention includes a substantially purified SECX polypeptide, e.g., any of the SECX polypeptides encoded by a SECX nucleic acid, and fragments, homologs, analogs, and derivatives thereof. The invention also includes a pharmaceutical composition that includes a SECX polypeptide and a pharmaceutically acceptable carrier or diluent.

In a still a further aspect, the invention provides an antibody that binds specifically to a SECX polypeptide. The antibody can be, e.g., a monoclonal or polyclonal antibody, and fragments, homologs, analogs, and derivatives thereof. The invention also includes a pharmaceutical composition including SECX antibody and a pharmaceutically acceptable carrier or diluent. The invention is also directed to isolated antibodies that bind to an epitope on a polypeptide encoded by any of the nucleic acid molecules described above.

The invention also includes kits comprising any of the pharmaceutical compositions described above.

The invention further provides a method for producing a SECX polypeptide by providing a cell containing a SECX nucleic acid, e.g., a vector that includes a SECX nucleic acid, and culturing the cell under conditions sufficient to express the SECX polypeptide encoded by the nucleic acid. The expressed SECX polypeptide is then recovered from the cell. Preferably, the cell produces little or no endogenous SECX polypeptide. The cell can be, e.g., a prokaryotic or eukaryotic cell.

The invention is also directed to methods of identifying a SECX polypeptide or nucleic acids in a sample by contacting the sample with a compound that specifically binds to the polypeptide or nucleic acid, and detecting complex formation, if present.

The invention further provides methods of identifying a compound that modulates the activity of a SECX polypeptide by contacting SECX polypeptide with a compound and determining whether the SECX polypeptide activity is modified.

The invention is also directed to compounds that modulate SECX polypeptide activity identified by contacting a SECX polypeptide with the compound and determining whether the compound modifies activity of the SECX polypeptide, binds to the SECX polypeptide, or binds to a nucleic acid molecule encoding a SECX polypeptide.

In a another aspect, the invention provides a method of determining the presence of or predisposition of a SECX-associated disorder in a subject. The method includes providing a sample from the subject and measuring the amount of SECX polypeptide in the subject sample. The amount of SECX polypeptide in the subject sample is then compared to the amount of SECX polypeptide in a control sample. An alteration in the amount of SECX polypeptide in the subject protein sample relative to the amount of SECX polypeptide in the control protein sample indicates the subject has a tissue proliferation-associated condition. A control sample is preferably taken from a matched individual, i.e., an individual of similar age, sex, or other general condition but who is not suspected of having a tissue proliferation-associated condition. Alternatively, the control sample may be taken from the subject at a time when the subject is not suspected of having a tissue proliferation-associated disorder. In some embodiments, the SECX is detected using a SECX antibody.

In a further aspect, the invention provides a method of determining the presence of or predisposition of a SECX-associated disorder in a subject. The method includes providing a nucleic acid sample, e.g., RNA or DNA, or both, from the subject and measuring the amount of the SECX nucleic acid in the subject nucleic acid sample. The amount of SECX nucleic acid sample in the subject nucleic acid is then compared to the amount of a SECX nucleic acid in a control sample. An alteration in the amount of SECX nucleic acid in the sample relative to the amount of SECX in the control sample indicates the subject has a tissue proliferation-associated disorder.

In a still further aspect, the invention provides method of treating or preventing or delaying a SECX-associated disorder. The method includes administering to a subject in which such treatment or prevention or delay is desired a SECX nucleic acid, a SECX polypeptide, or a SECX antibody in an amount sufficient to treat, prevent, or delay a tissue proliferation-associated disorder in the subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Other features and advantages of the invention will be apparent from the following detailed description and claims.

### BRIEF DESCRIPTION OF THE FIGURES

FIG. 1 is a representation of a SEC1 nucleic acid sequence (SEQ ID NO:1), along with an amino acid sequence (SEQ ID NO:2) encoded by the nucleic acid sequence.

FIG. 2 is a representation of a SEC2 nucleic acid sequence (SEQ ID NO:3) according to the invention, along with an amino acid sequence (SEQ ID NO:4) encoded by the nucleic acid sequence.

FIG. 3 is a representation of a SEC3 nucleic acid sequence (SEQ ID NO:5) according to the invention, along with an amino acid sequence (SEQ ID NO:6) encoded by the nucleic acid sequence.

FIG. 4 is a representation of a SEC4 nucleic acid sequence (SEQ ID NO:7) according to the invention, along with an amino acid sequence (SEQ ID NO:8) encoded by the nucleic acid sequence.

FIG. 5 is a representation of a SEC5 nucleic acid sequence (SEQ ID NO:9) according to the invention, along with an amino acid sequence (SEQ ID NO:10) encoded by the nucleic acid sequence.

FIG. 6 is a representation of a SEC6 nucleic acid sequence (SEQ ID NO:11 ) according to the invention, along with an amino acid sequence (SEQ ID NO:12) encoded by the nucleic acid sequence.

FIG. 7 is a representation of a SEC7 nucleic acid sequence (SEQ ID NO:13) according to the invention, along with an amino acid sequence (SEQ ID NO:14) encoded by the nucleic acid sequence.

FIG. 8 is a representation of a SEC8 nucleic acid sequence (SEQ ID NO:15) according to the invention, along with an amino acid sequence (SEQ ID NO:16) encoded by the nucleic acid sequence.

FIG. 9 is a representation of a SEC9 nucleic acid sequence (SEQ ID NO:17) according to the invention, along with an amino acid sequence (SEQ ID NO:18) encoded by the nucleic acid sequence.

FIG. 10 is a representation of a SEC10 nucleic acid sequence (SEQ ID NO:19) according to the invention, along with an amino acid sequence (SEQ ID NO:20) encoded by the nucleic acid sequence.

FIG. 11 is a representation of a SEC11 nucleic acid sequence (SEQ ID NO:21) according to the invention, along with an amino acid sequence (SEQ ID NO:22) encoded by the nucleic acid sequence.

FIG. 12 is a representation of a SEC12 nucleic acid sequence (SEQ ID NO:23) according to the invention, along with an amino acid sequence (SEQ ID NO:24) encoded by the nucleic acid sequence.

FIG. 13 is a comparison of the amino acid sequence of an SEC12 polypeptide (SEQ ID NO:24) (“2096375-0-104”) of the invention and an SEC8 polypeptide (SEQ ID NO:16) (“2093675.0.1”) of the invention.

FIG. 14 is a representation of an alignment of the amino acid sequence of a SEC5 polypeptide encoded by clone 1795045.0.61 (SEQ ID NO:9) and the amino acid sequence of a SEC10 polypeptide encoded by clone 1795045.0.77 (SEQ ID NO:19)

FIG. 15 is a representation of an alignment of the semaphorin-like amino acid sequences of a SEC6 polypeptide (“20422974.0.132”) a SEC7 polypeptide (“20422974_{—}2”), and a SEC 11 polypeptide “20422974.0.132-ext2”, together with Q64151 and Q92854, two previously described semaphorins.

FIG. 16 is a representation of a Western blot of the SEC1; 3445452 protein (SEQ ID NO:2) secreted by 293 cells.

FIG. 17 represents a Western blot of the SEC2; 4011999 protein (SEQ ID NO:4) secreted by 293 cells.

FIG. 18 represents a Western blot of the SEC10; 1795045 protein (SEQ ID NO:20) secreted by 293 cells.

FIG. 19 is a representation of an expression analysis of various SECX sequences according to the invention.

TABLE 1

| | | | | | | Calculated | | | | |
|---|---|---|---|---|---|---|---|---|---|---|
| | | | | | | Molecular | Protein Similarity | | | |
| | | | | | | Weight of | (BLASTP Non- | | | |
| | | Tissue | Length | ORF | Amino Acid | Encoded | Redundant Composite | Protein Similarity | Signal Peptide | |
| SECX | Clone Number | Expreession | (nt) | (nt) | Length | Protein | Database) | (Human Sequence) | Cleavage Site (nt) | Cellular Localization |
| 1 | 3445452 | Prostate | 932 | 113-796 | 227 | 25734.1 | Identities 52/128 (40%), | Identities 44/120 (36%), | yyyy. Most likely | Outside − Cert. = |
| | | Gland | | | | | Positives 72/128 (56%) | Positives 66/120 (55%) | cleavage site | 0.7380. Appears to |
| | | | | | | | with ACC: P31044 | with ACC: P31044 | between positions | possess a cleavable |
| | | | | | | | Phosphatidylethanolamin | Phosphatidylethanolamin | 22 and 23: VTG- | N-terminal Signal |
| | | | | | | | e-Binding Protein | e-Binding Protein | DE. | Sequence. |
| | | | | | | | (PEBP); 23 Kd | (PEBP) Homo sapiens | | |
| | | | | | | | Morphine-Binding | 186 Amino Acid | | |
| | | | | | | | Protein (P23K) Rattus | residues. | | |
| | | | | | | | norvegicus. 187 amino acid residues | | | |
| 2 | 4011999 | Not Known | 734 | 66-(?)735 | 223 | 24499 | Identities 55/76 (72%), | Identities 48/127 (37%), | yyyy Most likely | Plasma Membrane − |
| | | | | | | | Positives 61/76 (80%) | Positives 69/127 (54%) | cleavage site | Cert. = 0.8056. Appears |
| | | | | | | | with ptnr SPTREMBL- | with ptnr: SPTREMBL- | between positions | to possess a cleavable |
| | | | | | | | ACC: Q13670 PMS2- | ACC: O75631 Uroplakin | 27 and 28. SLS- | N-terminal Signal |
| | | | | | | | Related Protein HPMSR6 | III Homo sapiens 287 | LD | Sequence. |
| | | | | | | | Homo sapiens 270 | amino acid residues | | |
| | | | | | | | amino acid residues. | | | |
| 3 | 17089878 | Fetal Brain | 2762 | 264-2630 | 788 | 88337 | Identities 729/788 (92%); | Identities 577/790 (73%); | yyyy. Most likely | Plasma Membrane − |
| | .0.5 | | | | | | Positives 758/788 (96%) | Positives 676/790 (85%) | cleavage site | Cert = 0.4600. Appears |
| | | | | | | | with ACC: P79995 | with ACC: P55285 | between positions | to possess a cleavable |
| | | | | | | | Cadherin-10 Precursor | Cadherin-6 Precursor | 22 and | N-terminal Signal |
| | | | | | | | Gallus gallus. 789 amino | (Kidney-Cahedrin) Homo | 23: CSECX-EI. | Sequence. |
| | | | | | | | acid residues Identities | sapiens 790 amino acid residues | | |
| | | | | | | | 636/650 (97%); Positives | | | |
| | | | | | | | 645/650 (99%) with rat | | | |
| | | | | | | | cadherin-10. 653 amino | | | |
| | | | | | | | acid residues. | | | |
| 4 | 17089878 | Fetal Brain | 1820 | 285-1704 | 473 | 52922.6 | Identities 445/473 (94%); | Identities 346/476 (72%); | | Plasma Membrane − |
| | .0.6 | | | | | | Positives 465/473 (98%) | Positives 415/476 (87%) | | Cert. = 0.7000. |
| | | | | | | | with ACC: P7995 789 aa | with ACC: P55285, | | Apparently lacks |
| | | | | | | | Cadherin-10 Precursor | human Cadherin-6 | | cleavable N-terminal |
| | | | | | | | | precursor Precursor (790 | | |
| | | | | | | | | aa) | | Signal Sequence. |
| | | | | Open | | | Protein Similarity | | | |
| | | | Nucleo- | Reading | | Calculated | (BLASTP Non- | | | |
| SEC | | Tissue | tide | Frame | Amino Acid | Molecular | Redundant Composite | Protein Similarity | Signal Peptide | |
| No. | Clone Number | Expreession | Length | (nt) | Length | Weight | Database) | (Human Sequence) | Cleavage Site (nt) | Cellular Localization |
| 5 | 1795045. | Brain, | 1508 | 226-1461 | 411 | 46054.5 | Identities 51/198 (25%), | Identities 51/198 (25%), | | Cytoplam − |
| | 0.61 | Thalamus, | | | | | Positives 71/198 (35%) | Positives 71/198 (35%) | | Cert. = 0.4500. Appears |
| | | Pituitary | | | | | with ACC: O00276 | with ACC: O00276 | | to possess no cleavable |
| | | Gland | | | | | Lymphocyte-Associated | Lymphocyte-Associated | | N-terminal Signal |
| | | | | | | | Receptor of Death 2 | Receptor of Death 2 | | Sequence |
| | | | | | | | Homo sapiens. 510 | Homo sapiens. 510 | | |
| | | | | | | | amino acid residues | amino acid residues. | | |
| 6 | 20422974 | Lymphoid | 2155 | 166-1938 | 590 | 66532.5 | Identities 497/582 (85%); | Identities 247/506 (48%), | yyyy. Most likely | Microbody |
| | .0.132 | Tissue | | | | | Positives 536/582 (92%) | Positives 330/506 (65%) | cleavage site | (Peroxisome) − |
| | | | | | | | with ACC: Q64151 | with ACC Q92854 | between positions | Cert. = 7480. Appears to |
| | | | | | | | Semaphorin 1 (M-SEMA | Semaphorin Homo | 20 and 21: GIG- | possess a cleavable |
| | | | | | | | FA Factor in Neural | sapiens. 862 Amino | AE. | N-terminal Signal |
| | | | | | | | Network Development) | Acid residues | | Sequence. |
| | | | | | | | Mus musculus. 834 | | | |
| | | | | | | | amino acid residues. | | | |
| 7 | 20422974_2 | Lymphoid | 2284 | 166-1956 | 596 | 66969.8 | Identities 498/585 (85%); | Identities 265/558 (47%), | yyyy. Most likely | Microbody |
| | | Tissue | | | | | Positives 540/585 (92%) | Positives 353/558 (63%) | cleavage site | (Peroxisome) − |
| | | | | | | | with ACC: Q64151 | with ACC: Q92854 | between positions | Cert. = 7480. Appears to |
| | | | | | | | Semaphorin 1 (M-SEMA | Semaphorin Homo | 20 and 21. GIG- | possess a cleavable |
| | | | | | | | FA Factor in Neural | sapiens. 862 Amino | AE. | N-terminal Signal |
| | | | | | | | Network Development) | Acid residues | | Sequence |
| | | | | | | | Mus musculus 834 | | | |
| | | | | | | | amino acid residues | | | |
| 8 | 20936375 | Kidney | 1930 | 148-1758 | 536 | 60306.7 | Identities 453/531 (85%); | Identities 37/91 (40%), | nnny. Most likely | Plasma Membrane − |
| | 0.1 | | | | | | Positives 482/531 (90%) | Positives 58/91 (63%) | cleavage site | Cert. = 0.7000. Appears |
| | | | | | | | with ACC: P07106 | with ACC: O75521 DBI- | between positions | to possess a cleavable |
| | | | | | | | Bovine DBI-Related | Related Protein Homo | 15 and 16: SWC- | N-terminal Signal |
| | | | | | | | Brain Membrane Protein. | sapiens 364 amino acid | CC. | Sequence |
| | | | | | | | | residues | | |
| 9 | 20936785 | Brain, Fetal | 930 | 123-626 | 167 | 18440 | Identities 167/167 | Identities 167/167 | nnny. Most likely | Plasma Membrane − |
| | .0.1 | Brain | | | | | (100%) with Human | (100%) with Human | cleavage site | Cert. = 64000. Appears |
| | | | | | | | Transmembrane Protein | Transmembrane Protein | between positions | to possess an |
| | | | | | | | HTMPN-46. | HTMPN-46. | 31 and 32 TPR- | uncleavable N-terminal |
| | | | | | | | | | LS. | Signal Sequence. |
| | | | | | | | | | | Likely a Type IIIa |
| | | | | | | | | | | Membrane Protein |
| | | | | Open | | | Protein Similarity | | | |
| | Clone | | Nucleo- | Reading | | Calculated | (BLASTP Non- | | | |
| SEC | Identification | Tissue | tide | Frame | Amino Acid | Molecular | Redundant Composite | Protein Similarity | Signal Peptide | |
| No. | Number | Expreession | Length | (nt) | Length | Weight | Database) | (Human Sequence) | Cleavage Site (nt) | Cellular Localization |
| 10 | 1795045 | Brain, | 1737 | 296-1690 | 464 | 51645.6 | Identities 51/198 (25%), | Identities 51/198 (25%); | | Cytoplam − |
| | .0.77 | Thalamus | | | | | Positives 71/198 (35%) with | Positives 71/198 (35%) with | | Cert. = 0.4500. |
| | | | | | | | ACC: O00276 Lymphocyte- | ACC: O00276 Lymphocyte- | | Appears to possess no |
| | | | | | | | Associated Receptor of Death | Associated Receptor of | | cleavable N-terminal |
| | | | | | | | 2 Homo sapiens. 510 amino | Death 2 Homo sapiens. 510 | | Signal Sequence |
| | | | | | | | acid residues | amino acid residues. | | |
| 11 | 20422974.0.1 | Lymphoid Tissue, | 2156 | 166-2040 | 624 | 70478.1 | Identities 501/599 (83%), | Identities 501/599 (83%), | yyyy. Most likely | Microbody |
| | 32_ext2 | Aorta, Breast, | | | | | Positivies 542/599 (90%) with | Positives 542/599 (90%) | cleavage site | (Peroxisome) − |
| | | Colon, Foreskin, | | | | | ACC: Q92854 Semaphorin | With ACC: Q92854 | between positions | Cert. = 7480. Appears |
| | | Germ Cell, | | | | | Homo sapiens. 862 Amino | Semaphorin Homo sapiens. | 20 and 21: | to possess a cleavable |
| | | Muscle, | | | | | Acid residues. | 862 Amino Acid residues. | GIG-AE. | N-terminal Signal |
| | | Prostate, Spleen, | | | | | | | | Sequence. |
| | | Stomach, and | | | | | | | | |
| | | Uterus. | | | | | | | | |
| 12 | 20936375.0.1 | Kidney | 1930 | 7-1611 | 534 | 60037.3 | Identities 453/531 (85%), | Identities 37/91 (40%), | | Plasma Membrane − |
| | 04 | | | | | | Positives 482/531 (90%) with | Positives 58/91 (63%) with | | Cert. = 0.7300. |
| | | | | | | | ACC: P07106 Bovine DBI- | ACC O75521 DBI-Related | | Appears not to possess |
| | | | | | | | Related Brain Membrane | Protein Homo sapiens 364 | | a cleavable N-terminal |
| | | | | | | | Protein. | amino acid residues | | Signal Sequence. |

### DETAILED DESCRIPTION OF THE INVENTION

The invention provides polynucleotides and the polypeptides encoded thereby. Included in the invention are 12 nucleic acid sequences and their encoded polypeptides. These sequences are collectively referred to as “SECX nucleic acids” or “SECX polynucleotides”, and the corresponding encoded polypeptide is referred to as a “SECX polypeptide” or “SECX protein”. Unless indicated otherwise, “SECX” includes SEC1, SEC2, SEC3, SEC4, SEC5, SEC6, SEC7, SEC8, SEC9, SEC 10, SEC 11 and SEC12.

TABLE 1 provides a summary of various disclosed SECX nucleic acids and their encoded polypeptides. The table includes the following features:

Column 1 of TABLE 1, entitled “SECX No.”, denotes a SECX number assigned to a nucleic acid according to the invention.

Column 2 of TABLE 1, entitled “Clone Identification number” provides a second identification number for the indicated SP.

Column 3 of TABLE 1, entitled “Tissue Expression”, indicates the tissue in which the indicated SECX nucleic acid is expressed.

Columns 4-7 of TABLE 1 describes structural information as indicated for the indicated SECX nucleic acids and polypeptides.

Column 8 of TABLE 1, entitled “Protein Similarity” lists previously described proteins from BLASTP Non-redundant Composite database that are related to polypeptides encoded by the indicated SECX. These sequences can be retrieved from http://vww.ncbi.nlm.nih.gov/.

Column 9 of TABLE 1, entitled “Protein Similarity” lists previously described Human Sequences that are related to polypeptides encoded by the indicated SECX.

Column 10 of TABLE 1, entitled “Signal Peptide Cleavage Site” indicates the putative nucleotide position where the signal peptide is cleaved as determined by SignalP.

Column 11 of TABLE 1, entitled “Cellular Localization” indicates the putative cellular localization of the indicated SECX polypeptides.

TABLE 2 includes clone identification numbers corresponding to various SECX sequences, as well as a Sequence Identification Number (SEQ ID NO:) for the disclosed SECX nucleic acids and polypeptides.

TABLE 2

| Clone Identification | | SEQ ID NO: | SEQ ID NO: |
|---|---|---|---|
| Number | SECX | (Nucleic Acid) | (Polypeptide) |
| 3445452 | SEC1 | 1 | 2 |
| 4011999 | SEC2 | 3 | 4 |
| 17089878.0.5 | SEC3 | 5 | 6 |
| 17089878.0.6 | SEC4 | 7 | 8 |
| 1795045.0.61 | SEC5 | 9 | 10 |
| 20422974.0.132 | SEC6 | 11 | 12 |
| 20422974.2 | SEC7 | 13 | 14 |
| 20936375.0.1 | SEC8 | 15 | 16 |
| 20936785.0.1 | SEC9 | 17 | 18 |
| 1795045.0.77 | SEC10 | 19 | 20 |
| 20422974.0.132-ext2 | SEC11 | 21 | 22 |
| 20936375.0.104 | SEC12 | 23 | 23 |
| SEC1 MatF | | 25 | |
| SEC1 Rev | | 26 | |
| PSec-V5-His Forward | | 27 | |
| PSec-V5-His Reverse | | 28 | |
| SEC2 F-Topo-Forward | | 29 | |
| SEC2 F-Topo-Reverse | | 30 | |
| SEC2 C-Forward | | 31 | |
| SEC2 SECR | | 32 | |
| SEC10 Forward | | 33 | |
| SEC10 Reverse | | 34 | |
| Ag 36 (F) | | 35 | |
| Ag 36 (R) | | 36 | |
| Ag 36 (P) | | 37 | |
| Ag 123 (F) | | 38 | |
| Ag 123 (R) | | 39 | |
| Ag 123 (P) | | 40 | |
| Ag80 (F) | | 41 | |
| Ag80 (R) | | 42 | |
| Ag80 (P) | | 43 | |
| Ag 37 (F) | | 44 | |
| Ag 37 (R) | | 45 | |
| Ag 37 (P) | | 46 | |
| Ag 174 (F) | | 47 | |
| Ag 174 (R) | | 48 | |
| Ag 174 (P) | | 49 | |

Nucleic acid sequences and polypeptide sequences for SECX nucleic acids and polypeptides, as disclosed herein, are provided below.

SECX nucleic acids, and their encoded polypeptides, according to the invention are useful in a variety of applications and contexts. For example, various SECX nucleic acids and polypeptides according to the invention are useful, inter alia, as novel members of the protein families according to the presence of domains and sequence relatedness to previously described proteins.

SECX nucleic acids and polypeptides according to the invention can also be used to identify cell types for an indicated SECX according to the invention. Non-limiting examples of such cell types are listed in TABLE 1, column 3 for a SECX according to the invention. Additional utilities for SECX nucleic acids and polypeptides, as disclosed herein, will be discussed, below.

### SEC1

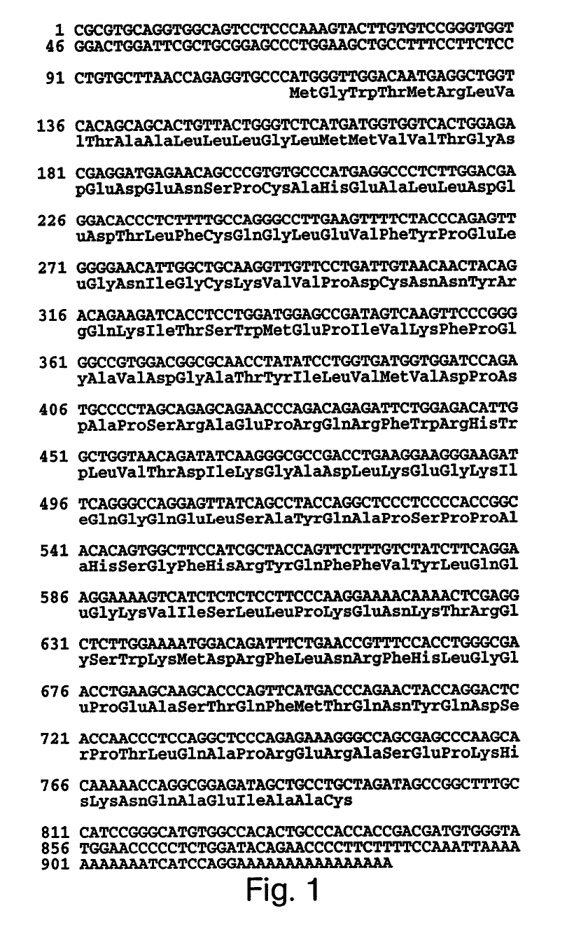

A SEC1 nucleic acid and polypeptide according to the invention includes the nucleic acid sequence of clone 3445452 (SEQ ID NO:1). The disclosed sequence is 932 nucleotides in length and contains an open reading frame (ORF) from nucleotides 113-794. The ORF encodes a secreted protein including 227 amino acid residues (SEQ ID NO:2) with a predicted molecular weight of 25734.1 daltons. The amino acid sequence of the disclosed protein is also shown in FIG. 1.

The disclosed SEC1 nucleic acid sequence was originally identified in prostate tissue.

The disclosed SEC1 polypeptide sequence is predicted by the PSORT computer program to localize to the outside of the plasma membrane with a certainty of 0.7380. The SignalP computer program predicts that there is a cleavable N-terminal Signal Sequence, with the most likely cleavage site between residues 22 and 23 in the sequence VTG-DE.

52 of 128 residues (40%) of the encoded polypeptide are identical to, and 72 of 128 residues (56%) are positive with, the 187 residue Rattus norvegicus phosphatidylethanolamine-binding protein (PEBP) (23 kDa morphine-binding protein) (P23K) (ACC: P31044).

The encoded protein also has 44 of 120 residues (36%) identical to, and 66 of 120 residues (55%) positive with, a 186 residue human phosphatidylethanolamine-binding protein (PEBP) (neuropolypeptide H3) (ACC:P30086). As a result of these similarities, a SEC1 protein of the invention includes a protein having membrane associated or membrane binding functions.

A SEC1 polypeptide includes the membrane-associated proteins of the invention encoded by the disclosed SEC1 nucleic acid sequence. as well as any mature protein arising therefrom as a result of post-translational modifications. Thus, the proteins of the invention encompass both a precursor and any active forms of the SEC1 protein.

### SEC2

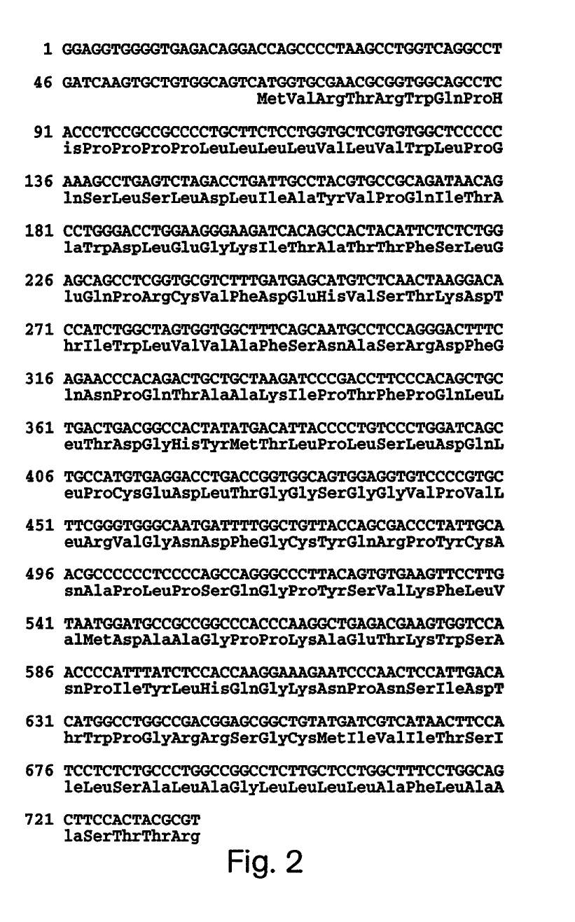

A SEC2 nucleic acid according to the invention includes the nucleic acid sequence (SEQ ID NO:3) of clone 4011999. The nucleic acid sequence is shown in FIG. 2. The disclosed nucleotide sequence includes 734 nucleotides. An ORF is present in the nucleotide sequence beginning with an initiation codon at nucleotides 66-68. No stop codon is present in the ORF.

The encoded protein is a secreted protein including 223 amino acid residues (SEQ ID NO:4), as shown in FIG. 2, with a predicted molecular weight of 24499 Daltons. The disclosed SEC2 polypeptide is predicted by the PSORT computer program to localize in Plasma Membrane with a certainty of 0.8056. The SignalP computer program predicts that the protein appears to possess a cleavable N-terminal signal sequence. A likely cleavage site is between residues 27 and 28, in the sequence SLS-LD.

The segment containing residues 79-153 of the disclosed SEC2 polypeptide has 55 of 76 amino acid residues (72%) identical to, and 61 of 76 residues (80%) positive with, the 270 amino acid residue human PMS2 related protein HPMSR6 (SPTREMBL-ACC:Q13670). This protein is described in Nicolaides et al., Genomics 30: 195-206, 1995.

The segment of the disclosed polypeptide containing residues 109-219 has 48 of 127 residues (37%) identical to, and 69 of 127 residues (54%) positive with, the 287 residue human uroplakin III (SPTREMBL-ACC:O75631), a cell surface glycoprotein that is differentiation-dependent.

Uroplakin III is 47 kDa tissue-specific and differentiation-dependent urothelial cell surface glycoprotein. See, Wu, et al., J. Cell. Sci. 106: 31-43, 1993. It has been recently demonstrated that a 47 kDa glycoprotein, uroplakin III (UPIII), in conjunction with uroplakins I (27 kDa) and II (15 kDa), forms the asymmetric unit membrane (AUM), which is a highly specialized biomembrane characteristic of the apical surface of bladder epithelium. Deglycosylation and cDNA sequencing revealed that UPIII contains up to 20 kDa of N-linked sugars attached to a core protein of 28.9 kDa. The presence of an N-terminal signal peptide sequence and a single transmembrane domain located near the carboxyl-terminus, plus the amino-terminal location of all the potential N-glycosylation sites, points to a type I (i.e., N-exo/C-cyto) membrane spanning configuration. Thus, the mass of the extracellular domain (20 kDa plus up to 20 kDa of sugar) of UPIII greatly exceeds that of its intracellular domain (5 kDa). Such an asymmetrical mass distribution, which is a feature shared by other major uroplakins, provides a molecular explanation as to why the luminal leaflet of AUM is almost twice as thick as the cytoplasmic one. The fact that only UPIII among the major AUM proteins possesses a significant cytoplasmic domain suggests that this molecule may play an important role in AUM-cytoskeleton interaction in terminally-differentiated urothelial cells.

Proteins of the invention include polypeptides having the amino acid sequence encoded by the disclosed SEC2 nucleic acid, as well as any mature protein arising therefrom as a result of post-translational modifications. Thus, the proteins of the invention encompass both a precursor and any active forms of the SEC2 protein. A SEC2 protein of the invention includes a polypeptide having the functional activity of a uroplakin-like protein.

### SEC3

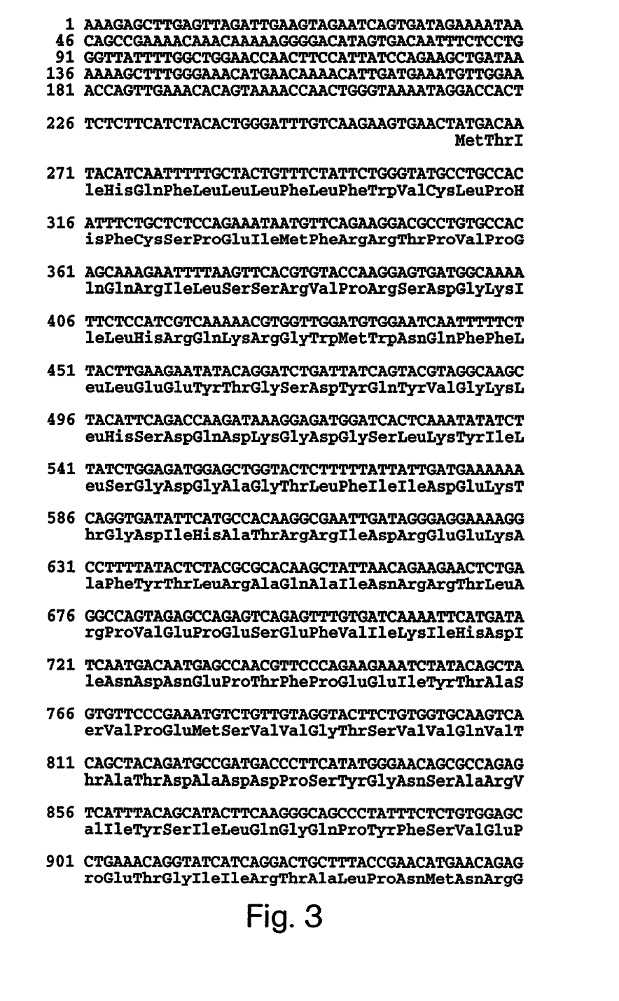

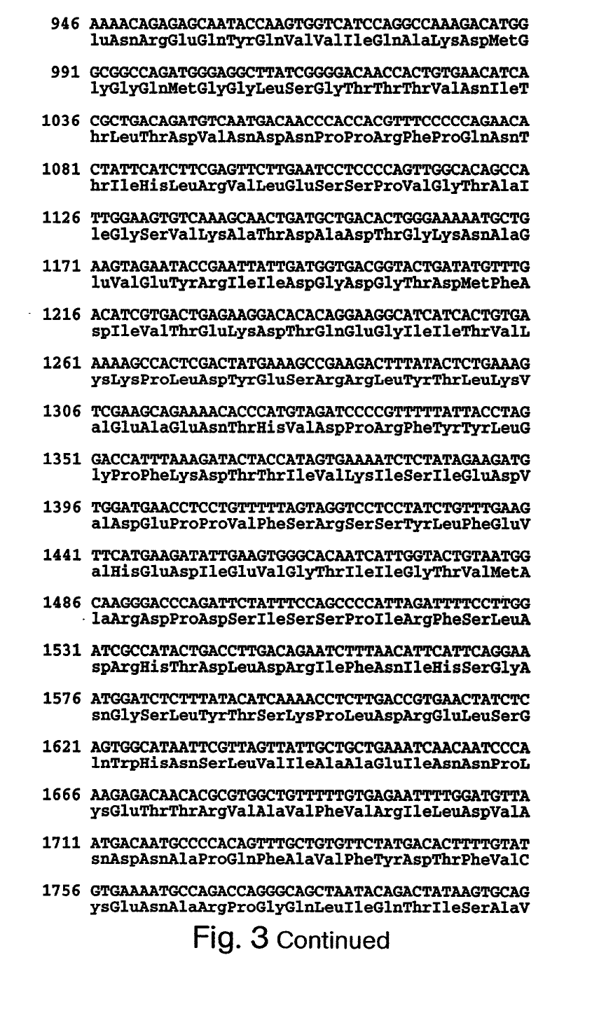

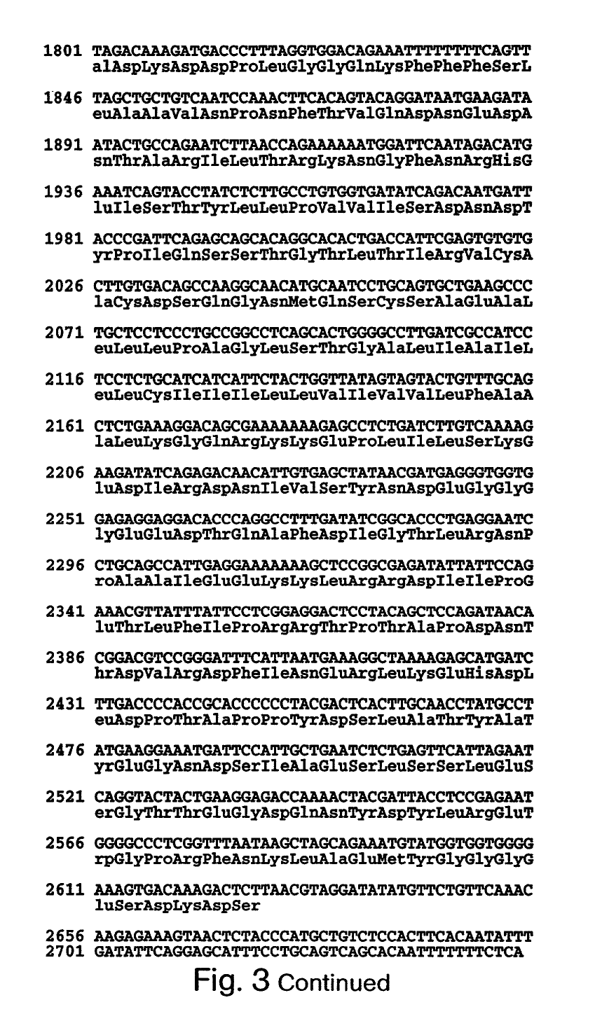

A SEC3 nucleic acid and polypeptide according to the invention includes the nucleic acid sequence (SEQ ID NO:5) of clone 17089878.0.5. The disclosed SEC3 nucleic acid sequence is 2672 nucleotides in length and is presented in FIG. 3. The sequence includes an ORF encompassing nucleotides 264-2630. The ORF encodes a secreted protein of 788 amino acid residues (SEQ ID NO:6) with a predicted molecular weight of 88337 daltons. The sequence of the encoded polypeptide is presented in FIG. 2.

Expression of the disclosed SEC3 nucleic acid is detected in salivary gland and in fetal brain tissue.

The encoded polypeptide is predicted by the PSORT computer program to localize to the plasma membrane with a certainty of 0.4600. The SignalP computer program predicts that there is a cleavable N-terminal Signal Sequence, with the most likely cleavage site between residues 22 and 23 in the sequence CSECX-EI.

The encoded protein encoded has 729 of 788 residues (92%) identical to, and 758 of 788 residues (96%) positive with, the 789 residue cadherin-10 precursor of Gallus gallus (chicken) (ACC:P79995). In addition, the SEC3 protein has 577 of 790 residues (73%) identical to, and 676 of 790 residues (85%) positive with, the 790 residue human cadherin-6 precursor (kidney-cadherin; K-cadherin) (ACC:P55285).

The encoded protein also has 636 of 650 residues (97%) identical to, and 645 of 650 residues (99%) positive with, rat cadherin-10. Rat cadherin-10 is a protein of 653 residues. See U.S. Pat. No. 5597725, and U.S. Pat. No. 5,646,250. SEC3 polypeptides of the invention therefore include novel members of the cadherin protein family.

Previously described cadherin family members include, e.g., rat and human cadherin-5, -8, -10, -11, -12, and -13. Cadherins are calcium-dependent cell adhesion proteins. They are glycosylated integral membrane proteins that have an amino-terminal extracellular domain (which determines binding specificity), a hydrophobic membrane spanning region, and a carboxyl-terminal cytoplasmic domain (which is highly conserved among members of the cadherin superfamily). The carboxyl-terminal domain interacts with the cytoskeleton through catenins and other cytoskeleton-associated proteins. Cadherin proteins may be used in the analysis of the role of cadherins in various cancers. Sequence analysis of the cadherin proteins also allows investigation of the structure and function of cadherin.

The cadherin proteins may be isolated using anti-cadherin antibodies. These antibodies may also be used to modulate the activity of cadherin, as well as to determine the tissue specific distribution of cadherin proteins. Each subclass of cadherins has a unique tissue distribution pattern.

SEC3 polypeptides of the invention include the polypeptide encoded by the disclosed SEC3 nucleic acid, as well as any mature protein arising therefrom as a result of post-translational modifications. Thus, the proteins of the invention encompass both a precursor and any active forms of the SEC3 protein.

### SEC4

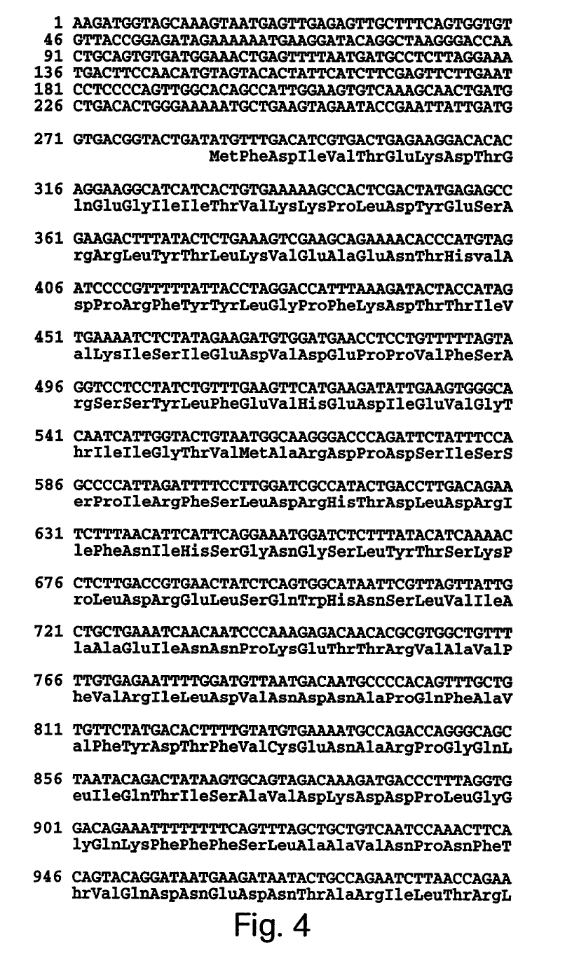

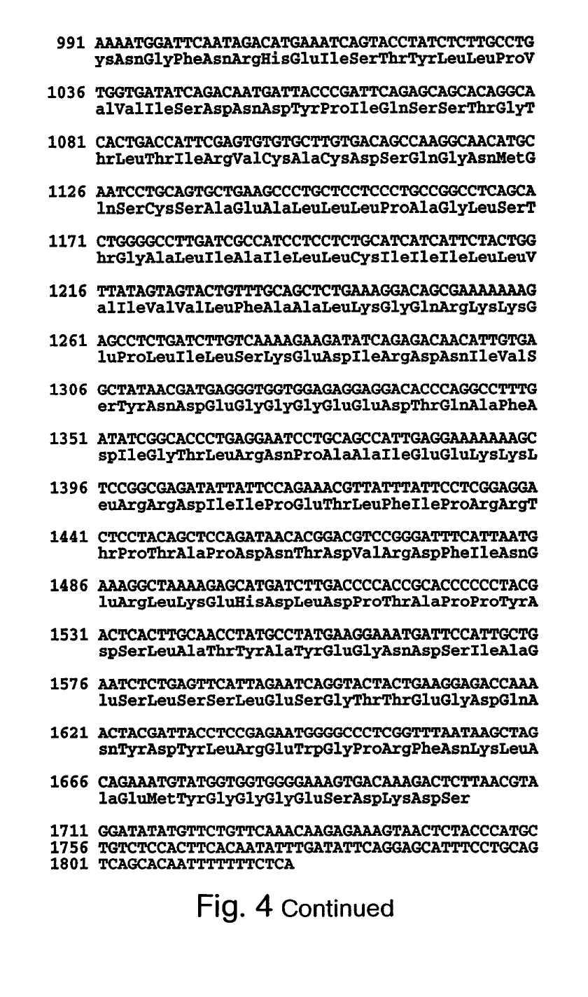

A SEC4 nucleic acid of the invention includes the nucleotide sequence (SEQ ID NO:7) of 17089878.0.6, which is shown in FIG. 4.

The nucleotide sequence of SEC4 includes 1820 basepairs, which contains an open reading frame from nucleotides 285-1706. The ORF encodes a polypeptide of 473 amino acid residues (SEQ ID NO:8),which has a molecular weight of 52922.6 Daltons. The sequence of the encoded polypeptide is also presented in FIG. 4.

The disclosed SEC4 polypeptide is predicted by the PSORT computer program to localize to the plasma membrane with a certainty of 0.7000, and does not appear to possess a cleavable N-terminal signal sequence.

The encoded polypeptide sequence (SEQ ID NO:8 appears to be a shortened form of the disclosed SEC3 (SEQ ID NO:6) protein. The encoded SEC4 polypeptide begins at amino acid residue 316 of the SEC3 protein terminates at an amino acid corresponding to the C-terminal amino acid residue of the SEC3 protein.

The disclosed SEC4 polypeptide has 445 of 473 residues (94%) identical to, and 465 of 473 residues (98%) positive with, the 789 residue cadherin-10 precursor from Gallus gallus (chicken) (ACC:P79995). In addition, the disclosed polypeptide has 346 of 476 residues (72%) identical to, and 415 of 476 residues (87%) positive with, the 790 residue human cadherin-6 precursor (kidney-cadherin) (K-cadherin) (ACC:P55285). SEC4 is therefore believed to represent a novel member of the cadherin protein family and may represent a splice-variant of SEC3.

The proteins of the invention encoded by SEC4 include the protein disclosed as being encoded by the ORF described herein, as well as any mature protein arising therefrom as a result of post-translational modifications. Thus, the proteins of the invention encompass both a precursor and any active forms of the SEC4 protein.

### SEC5

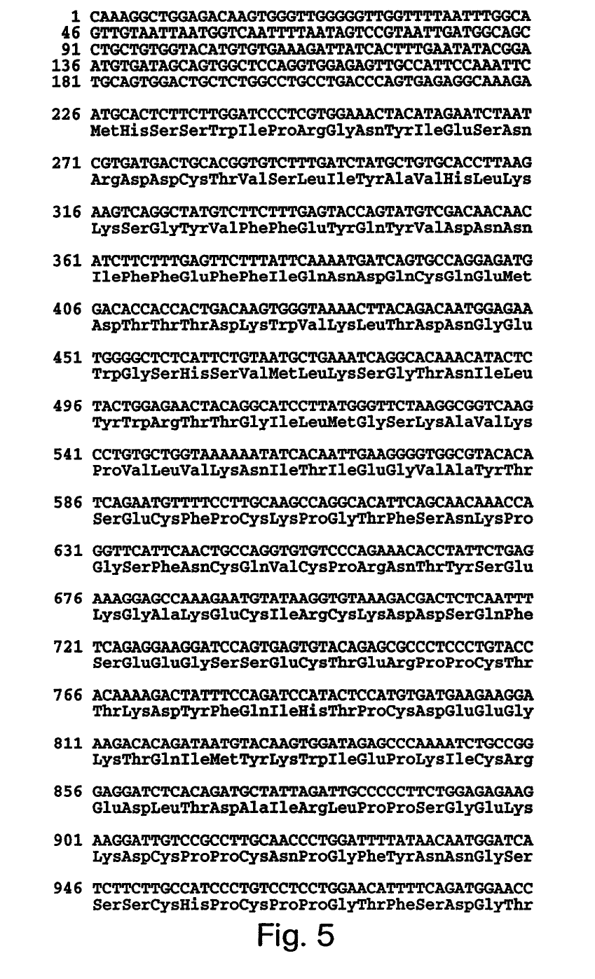

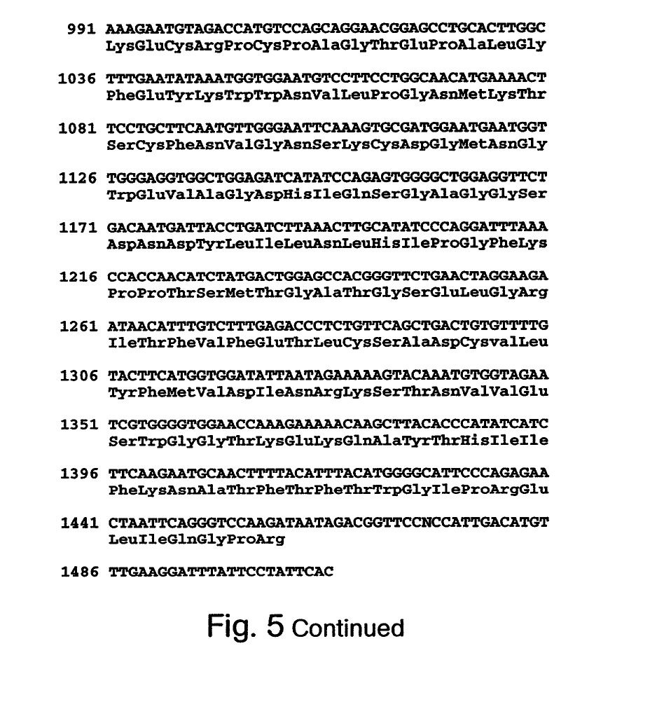

A SEC5 nucleic acid according to the invention includes the nucleic acid sequence (SEQ ID NO:9) of 1795045.0.61, which is shown in FIG. 5.

The disclosed nucleotide sequence includes 1508 nucleotides. An open reading frame (ORF) is present in the sequence encompassing nucleotides 226-1461. The ORF encodes a secreted protein of 411 amino acid residues (SEQ ID NO:10) with a predicted molecular weight of 46054.5 Daltons. The encoded polypeptide is predicted by the PSORT computer program to localize to the cytoplasm with a certainty of 0.4500 and does not appear to possess a cleavable N-terminal signal sequence.

The encoded polypeptide has 51 of 198 residues (25%) identical to, and 71 of 198 residues (35%) positive with, the 510 amino acid residue human lymphocyte-associated receptor of death 2(ACC:O00276).

SEC5 is expressed in the brain (in particular in the thalamus), the pituitary gland, and in 10 human total RNAs (brain, fetal brain, liver, fetal liver, skeletal muscle, pancreas, kidney, heart, lung & placenta).

The SEC5 proteins of the invention include the encoded SEC5 protein, as well as any mature protein arising therefrom as a result of post-translational modifications. Thus, the proteins of the invention encompass both a precursor and any active forms of the SEC5 protein.

### SEC6

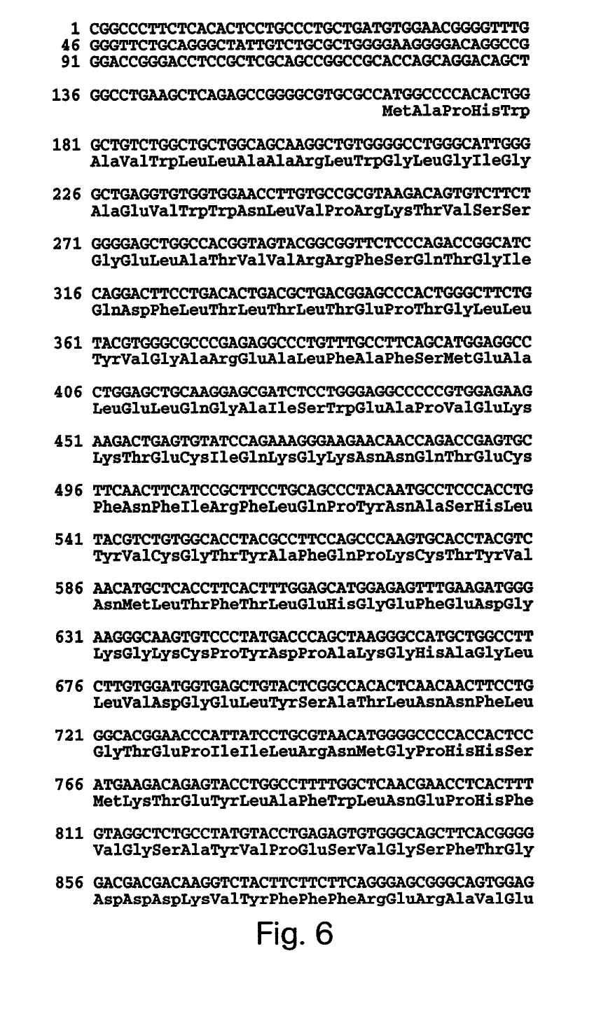

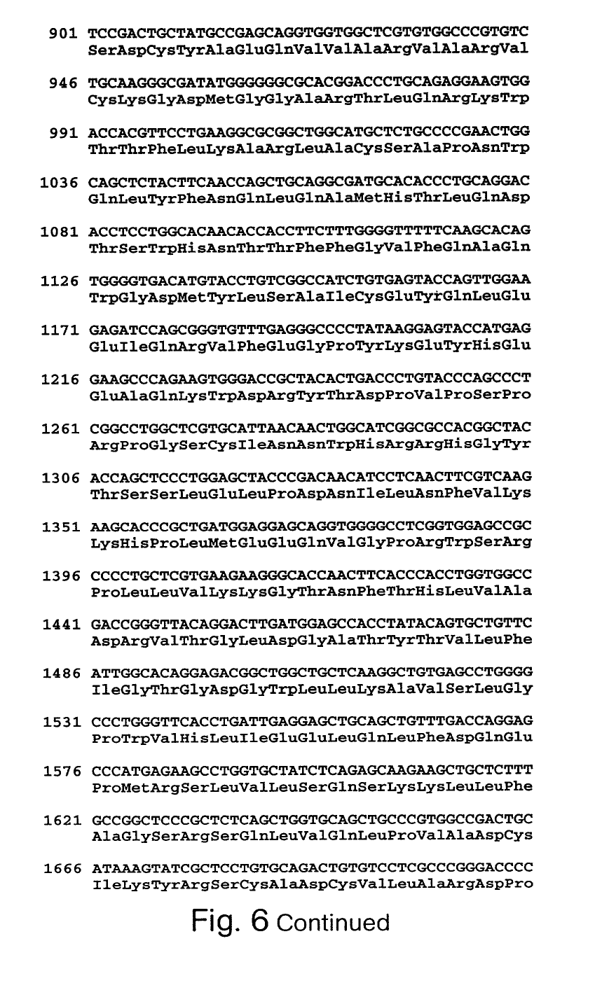

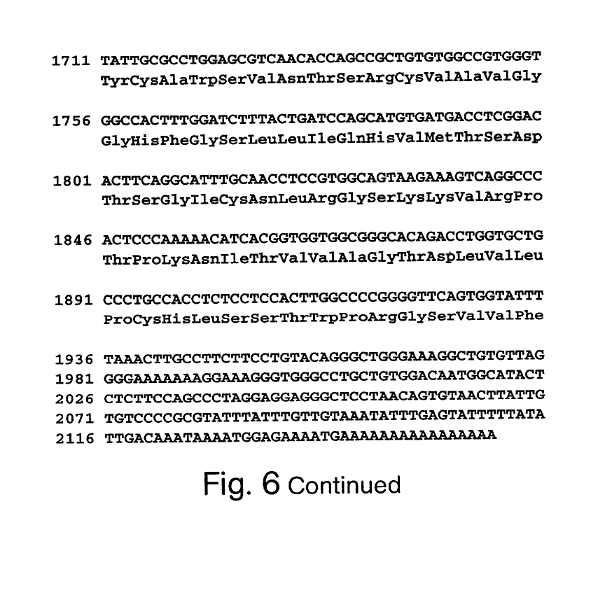

A SEC6 nucleic acid according to the invention includes the nucleic acid sequence (SEQ ID NO:11) of 204229740.132. The disclosed sequence is presented in FIG. 6. The sequence is 2155 nucleotides in length and includes an ORF spanning nucleotides 166-1938. The ORF encodes a secreted protein of 590 amino acid residues (SEQ ID NO:12). The encoded protein has a predicted molecular weight of 66532.5 Daltons.

The encoded polypeptide is predicted by the PSORT computer program to localize to the microbody (peroxisome) with a certainty of 0.7480. The SignalP computer program predicts that there is no cleavable N-terminal Signal Sequence, although the most likely cleavage site would appear to reside between residues 20 and 21 in the sequence GIG-AE.

The encoded polypeptide has 497 of 582 residues (85%) identical to, and 536 of 582 residues (92%) positive with, the 834 residue semaphorin I (M-SEMA FA factor in neural network development) from Mus musculus (mouse) (ACC:Q64151). In addition, the SEC6 protein has 247 of 506 residues (48%) identical to, and 330 of 506 residues (65%) positive with, the 862 residue human semaphorin (ACC:Q92854). Therefore, it is believed that SEC6 represents a novel human semaphorin.

Semaphorin was previously identified as CD100 (Hall, et al., Proc. Natl. Acad. Sci. U.S.A. 93(21): 11780-11785, 1996). The human leukocyte activation antigen CD100 is reported to be a semaphorin. Semaphorins have recently been described as neuronal chemorepellants that direct pioneering neurons during nervous system development. In addition, it has been demonstrated that CD100 induces B-cells to aggregate and improves their viability in vitro. These results suggest that semaphorins as exemplified by CD100 also play a functional role in the immune system. The novel human semaphorin-like proteins described herein have functional roles in the growth and/or differentiation of tissues of the immune system as well as analogous roles in other tissues of the body.

The SEC6 polypeptides of the invention include the disclosed SEC6 polypeptide, as well as any mature protein arising therefrom as a result of post-translational modifications. Thus, the proteins of the invention encompass both a precursor and any active forms of the SEC6 protein.

### SEC7

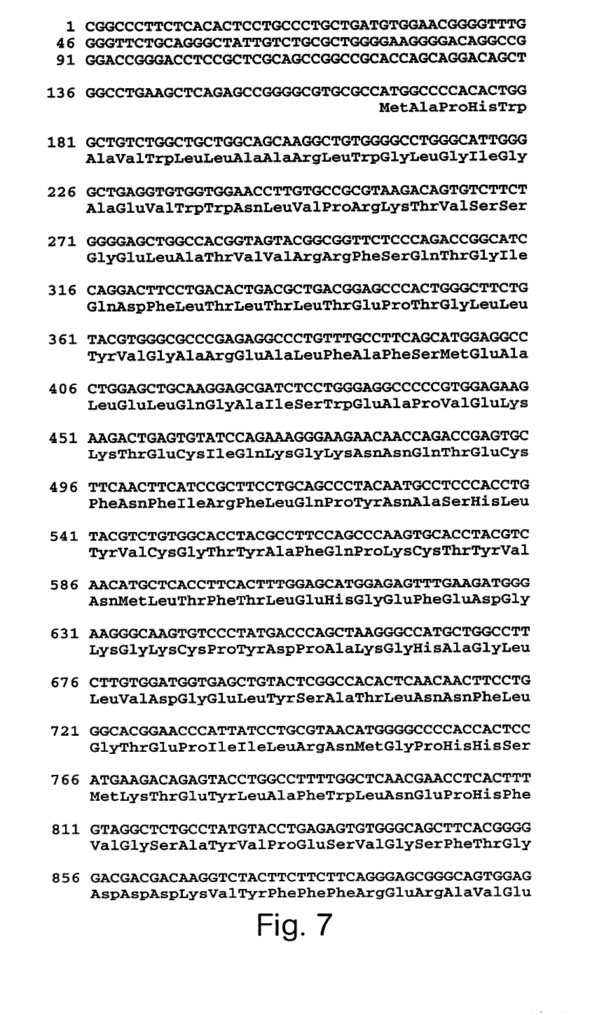

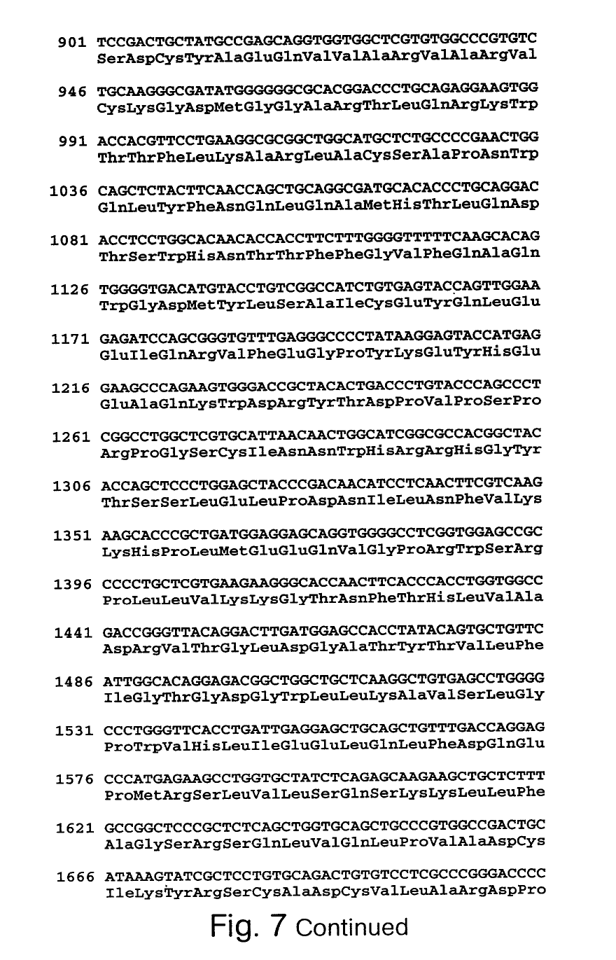

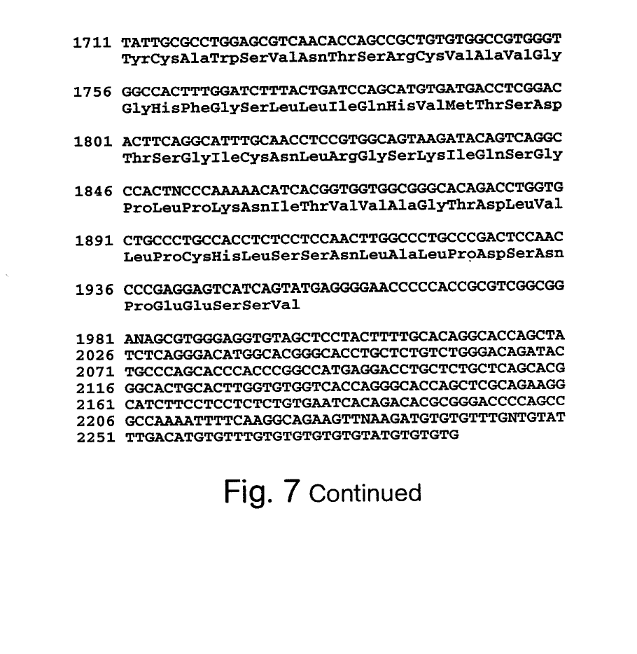

A SEC7 nucleic acid of the invention includes the nucleic acid sequence (SEQ ID NO:13) of clone 20422974_{—}2. The disclosed nucleotide sequence is presented in FIG. 7. The disclosed nucleotide sequence includes 2284 basepairs. An open reading frame (ORF) is present at nucleotides 166-1956. FIG. 7 also presents the amino acid sequence (SEQ ID NO:14) of the encoded protein. The encoded protein is a secreted protein that includes 596 amino acid residues and has a predicted molecular weight of 66969.8 Daltons.

The encoded polypeptide is predicted by the PSORT computer program to localize to the microbody (peroxisome) with a certainty of 0.7480. The SignalP computer program predicts that there is no cleavable N-terminal signal sequence, although the most likely cleavage site would appear to reside between residues 20 and 21 in the sequence GIG-AE.

The disclosed SEC7 protein has 498 of 585 residues (85%) identical to, and 540 of 585 residues (92%) positive with, the 834 residue semaphorin I (M-SEMA FA factor in neural network development) of Mus musculus (ACC:Q64151). Additionally the protein has 265 of 558 residues (47%) identical to, and 353 of 558 residues (63%) positive with, the 862 residue human semaphorin protein (ACC:Q92854). Therefore, it is believed that SEC7 represents a novel human semaphorin.

The proteins of the invention encoded by SEC7 include the protein disclosed as being encoded by the ORF described herein, as well as any mature protein arising therefrom as a result of post-translational modifications. Thus, the proteins of the invention encompass both a precursor and any active forms of the SEC7 protein.

The SEC7 polypeptides of the invention include the disclosed SEC7 polypeptide, as well as any mature protein arising therefrom as a result of post-translational modifications. Thus, the proteins of the invention encompass both a precursor and any active forms of the SEC7 protein.

### SEC8

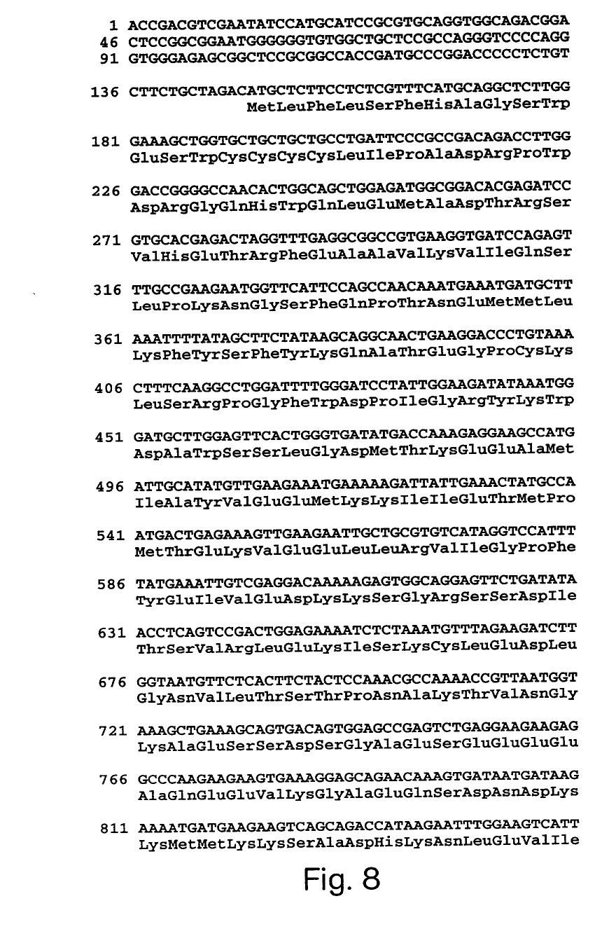

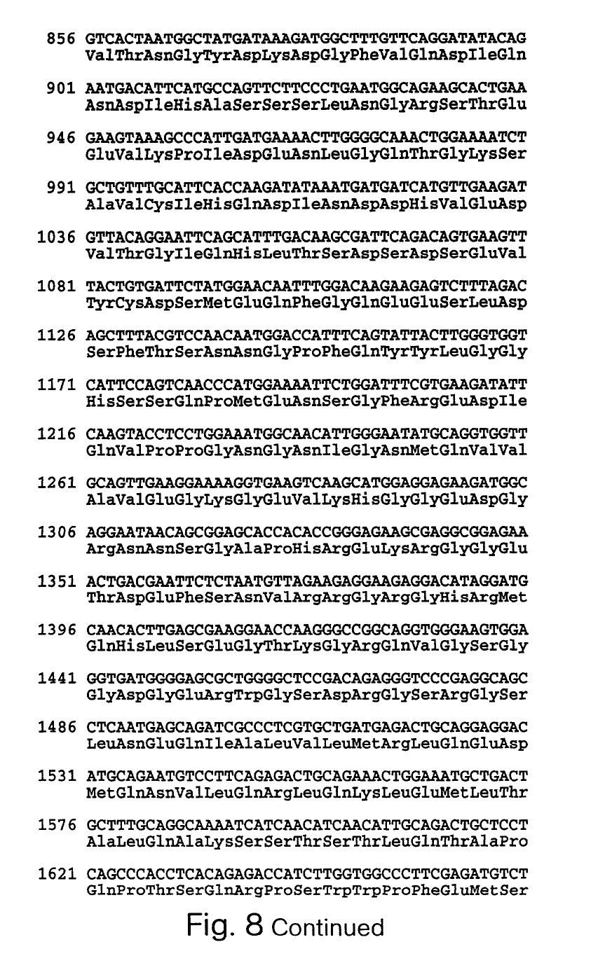

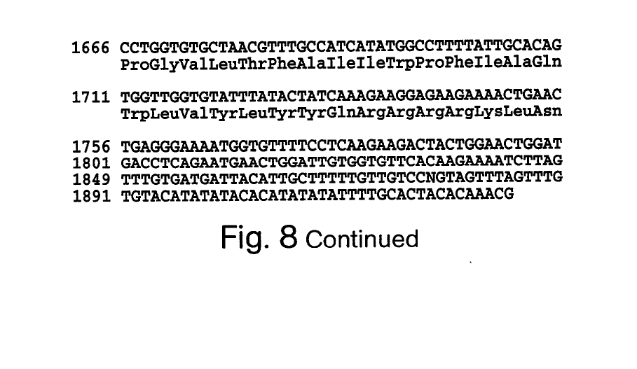

A SEC8 nucleic acid according to the invention includes the nucleic acid sequence (SEQ ID NO:15) of isolate 20936375.0.1. The sequence is shown in FIG. 8. The nucleotide sequence is 1930 basepairs and includes nucleotides an ORF from nucleotides 148-1758. The ORF encodes a secreted protein comprising 536 amino acid residues (SEQ ID NO:16), which is also presented in FIG. 8. The encoded protein has a predicted molecular weight of 60306.7 daltons and is predicted by the PSORT computer program to be localized to the Plasma Membrane with a certainty of 0.7000. The SignalP computer program predicts that there is no cleavable N-terminal Signal Sequence, although the most likely cleavage site would appear to reside between residues 15 and 16 in the sequence SWC-CC.

The encoded protein has 453 of 531 residues (85%) identical to, and 482 of 531 residues (90%) positive with, a bovine brain membrane protein with activity as a diazepam receptor or agonist(SWISSPROT-ACC:P07106). This bovine protein is described in WO8604239-A. In view of the origin of this clone from kidney, as well as the prediction that it is localized in the plasma membrane, it is likely that the encoded protein represents a receptor implicated in signaling pathways.

The SEC8 polypeptides of the invention include the disclosed SEC8 polypeptide, as well as any mature protein arising therefrom as a result of post-translational modifications. Thus, the proteins of the invention encompass both a precursor and any active forms of the SEC8 polypeptide.

### SEC9

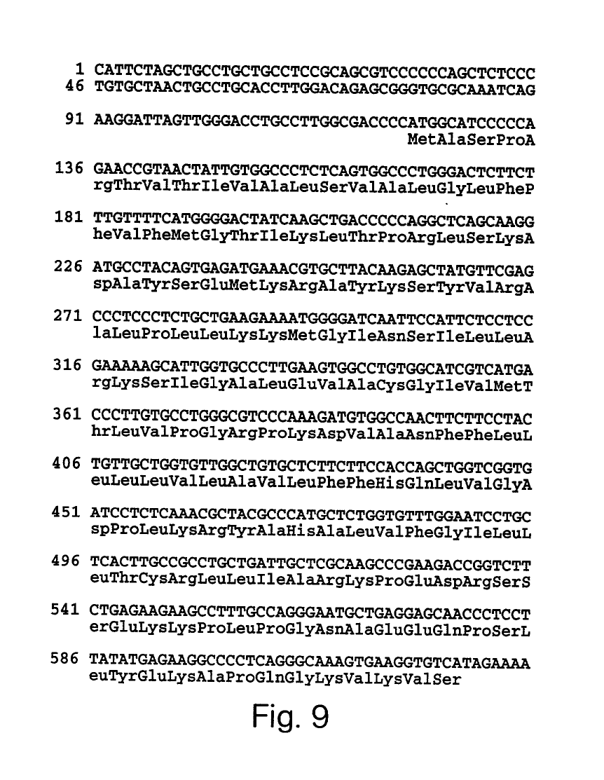

A SEC9 nucleic acid according to the invention includes the nucleic acid sequence of 20936785.0.1 (SEQ ID NO:17), which is shown in FIG. 9. The disclosed 630 nucleotide sequence includes an ORF from nucleotides 123-626. FIG. 9 also reveals that the ORF encodes a secreted protein that includes 167 amino acid residues (SEQ ID NO:18). The encoded protein has a predicted molecular weight of 18440 Daltons and is predicted by the PSORT computer program to be localized to the plasma membrane with a certainty of 0.6400. The SignalP computer program predicts that there is an uncleavable N-terminal Signal Sequence, although the most likely cleavage site would appear to reside between residues 31 and 32 in the sequence TPR-LS.

The SEC9 polypeptides of the invention include the disclosed SEC9 polypeptide, as well as any mature protein arising therefrom as a result of post-translational modifications. Thus, the proteins of the invention encompass both a precursor and any active forms of the SEC9 polypeptide.

### SEC10

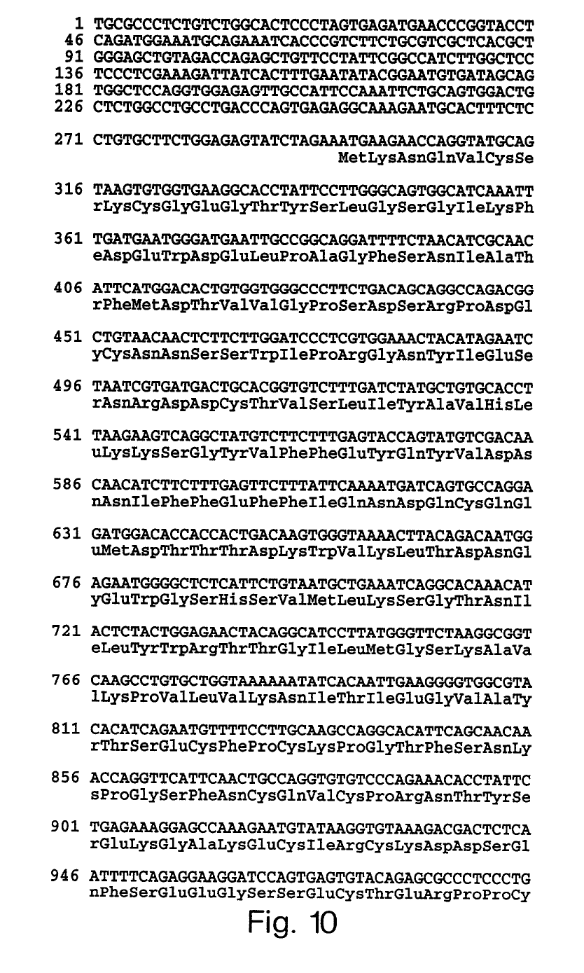

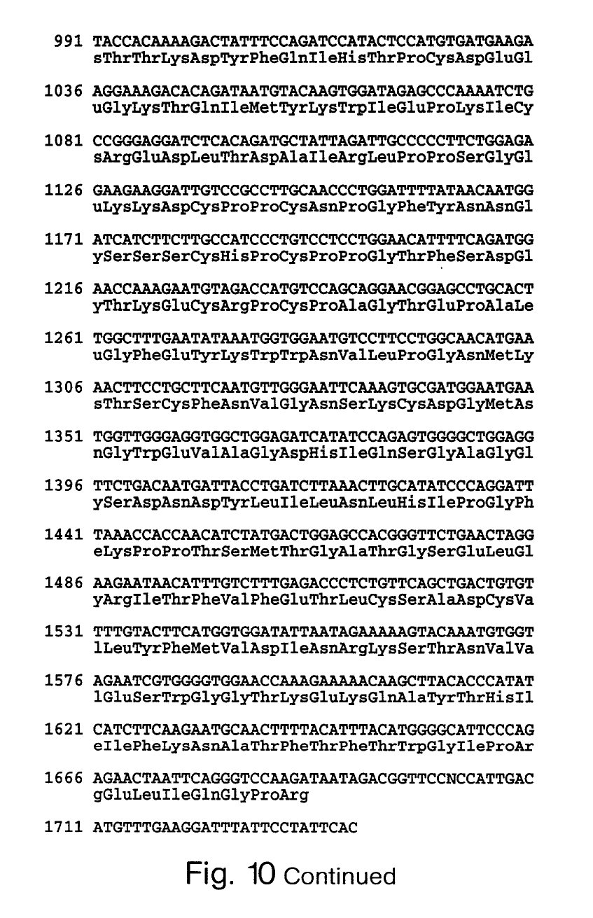

A SEC10 nucleic acid according to the invention includes the nucleic acid sequence (SEQ ID NO:19) shown in FIG. 10. The disclosed sequence is 1737 nucleotides and contains an open reading frame from nucleotides 296-1690. The open reading frame encodes a polypeptide of 464 amino acid residues (SEQ ID NO:10) with a predicted molecular weight of 51645.6 Daltons. The disclosed SEC10 nucleic acid is expressed in the brain, and in particular in the thalamus.

The encoded polypeptide is predicted by the PSORT computer program to localize to the cytoplasm with a certainty of 0.4500 and does not appear to possess a cleavable N-terminal signal sequence.

The encoded polypeptide has 51 of 198 residues (25%) identical to, and 71 of 198 residues (35%) positive with, the 510 amino acid residue human lymphocyte-associated receptor of death 2 (ACC:O00276).

The encoded SEC10 polypeptide is related to the disclosed SEC5 polypeptide. An alignment of the SEC5 protein (1795045.0.61) protein with the SEC10 protein is shown in FIG. 14. The alignment illustrates that: (i) the splice variant of SEC10 possesses an amino-terminal segment which contains an additional 53 residues; and (ii) the SEC5 and SEC10 sequences are identical beginning with the third amino acid residue of the overlapping region. The nucleic acid sequences of SEC5 (SEQ ID NO:9) and SEC10 (SEQ ID NO:19) differ in the 5′-untranslated regions included in the sequences illustrated in FIG. 5 and FIG. 10, as well as in that SEC5 lacks the segment encoding the region of the protein that is presumed to be removed by splicing.

The SEC10 polypeptides of the invention include the disclosed SEC10 polypeptide, as well as any mature protein arising therefrom as a result of post-translational modifications. Thus, the proteins of the invention encompass both a precursor and any active forms of the SEC10 polypeptide.

### SEC11

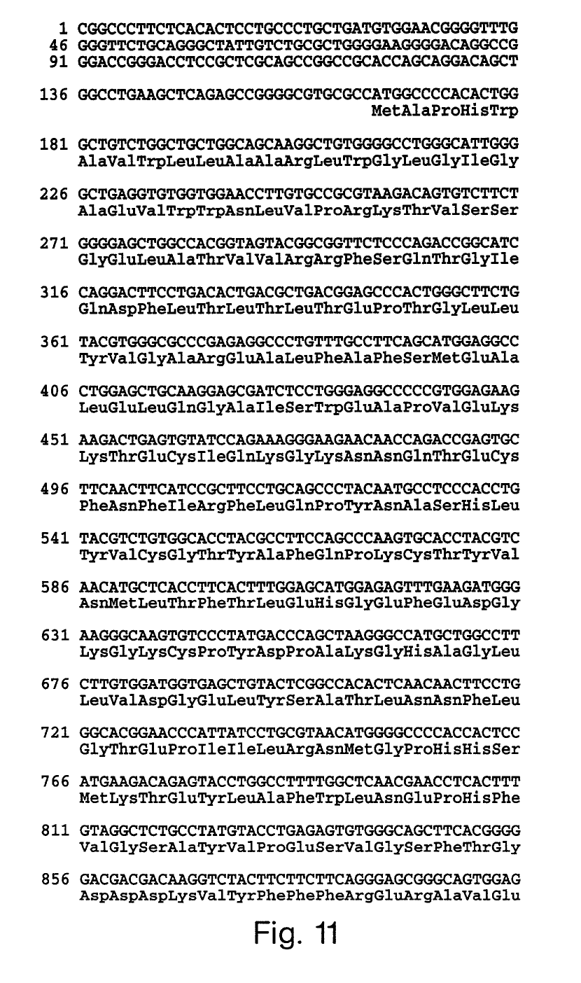

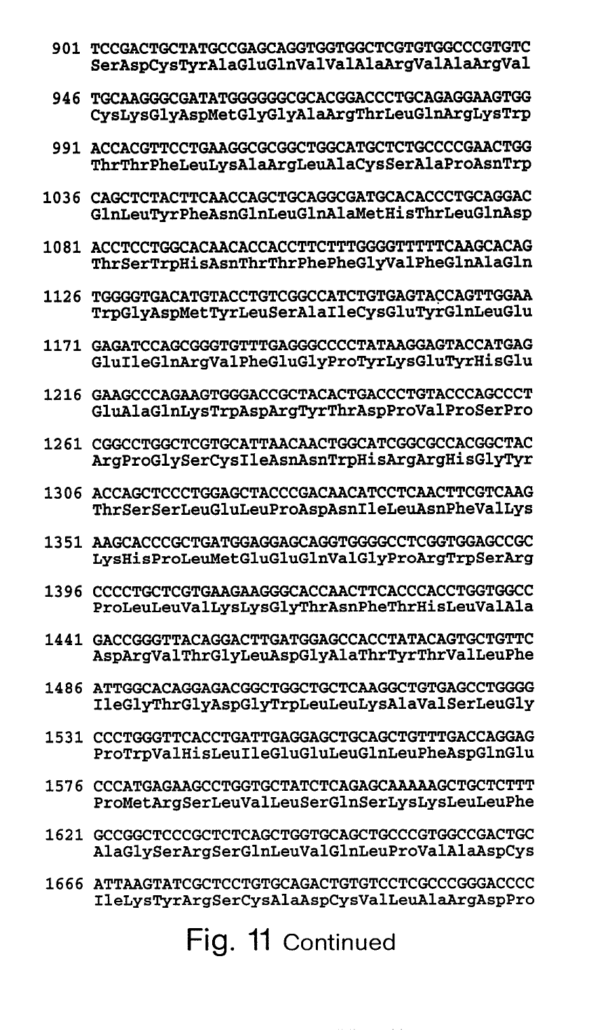

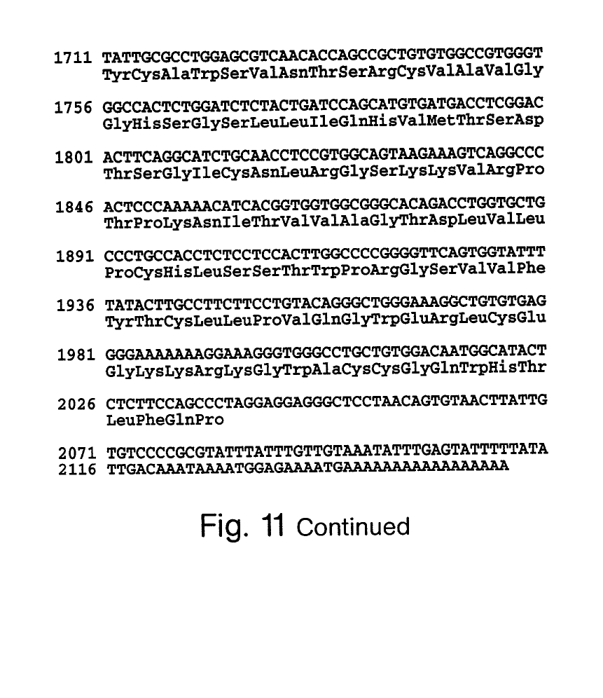

A SEC 11 nucleic acid according to the invention includes the nucleic acid sequence (SEQ ID NO:21) of 204229740.132_{—}132. FIG. 11 illustrates the disclosed SEC11 nucleic acid sequence. The disclosed nucleotide sequence 2156 nucleotides in length and includes an open reading frame (ORF) from nucleotides 166-2040. The ORF encodes a protein including 624 amino acid residues with a predicted molecular weight of 70478.1 Daltons. The polypeptide of SEC 11 protein is predicted by the PSORT computer program to localize to the Microbody (Peroxisome) with a certainty of 0.7480. The SignalP computer program predicts that there is no cleavable N-terminal Signal Sequence, although the most likely cleavage site would appear to reside between residues 20 and 21 in the sequence GIG-AE.

SEC11 has 501 of 599 residues (83%) identical to, and 542 of 599 residues (90%) positive with, the 834 residue semaphorin I (M-SEMA FA factor in neural network development)from Mus musculus (mouse) (ACC:Q64151). In addition, the SEC11 protein has 256 of 527 residues (48%) identical to, and 341 of 527 residues (64%) positive with the 862 residue human semaphorin (ACC:Q92854). Therefore, it is believed that SEC11 represents a novel human semaphorin. Semphorins have been described above.

The SEC11 polypeptides of the invention include the disclosed SEC11 polypeptide, as well as any mature protein arising therefrom as a result of post-translational modifications. Thus, the proteins of the invention encompass both a precursor and any active forms of the SEC11 polypeptide.

### SEC12

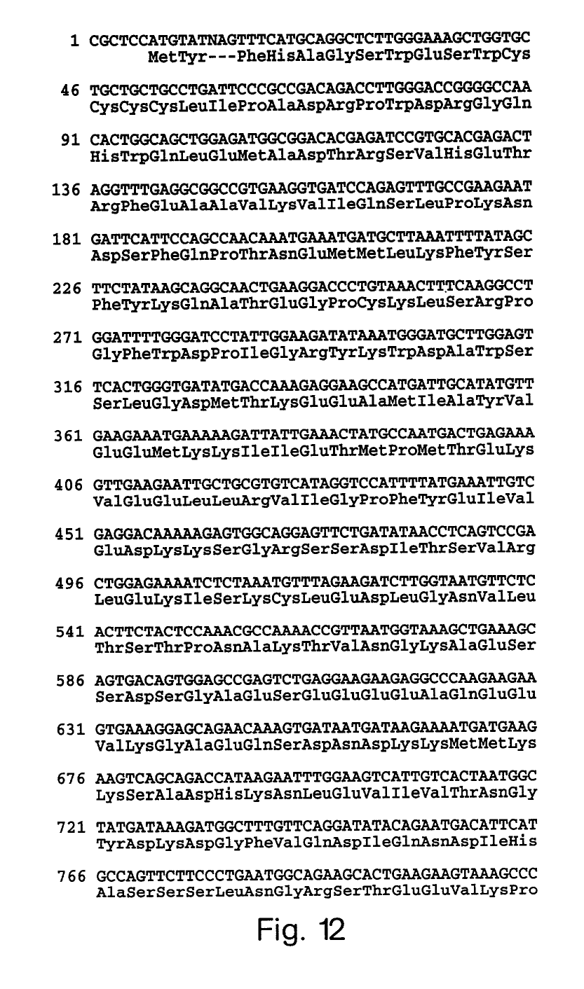

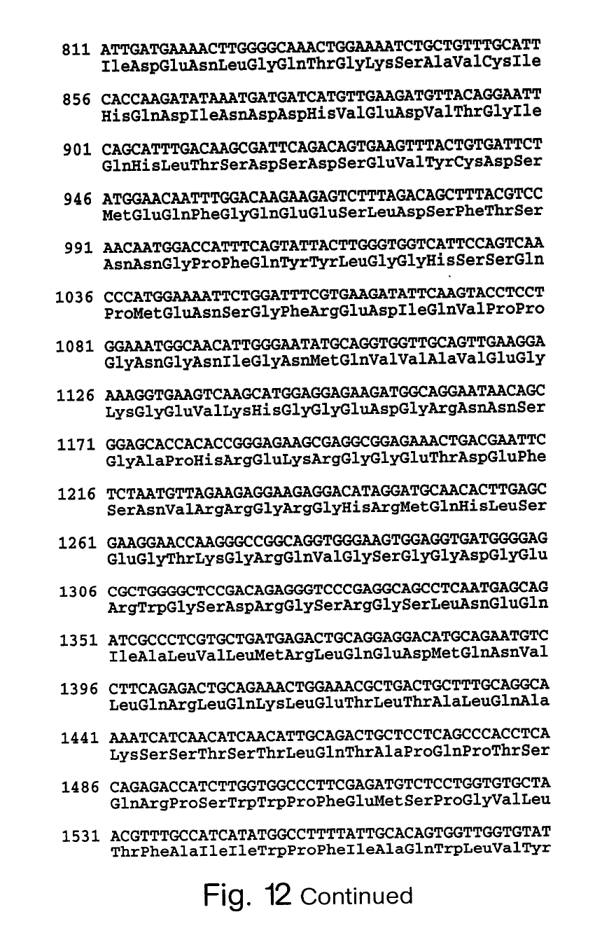

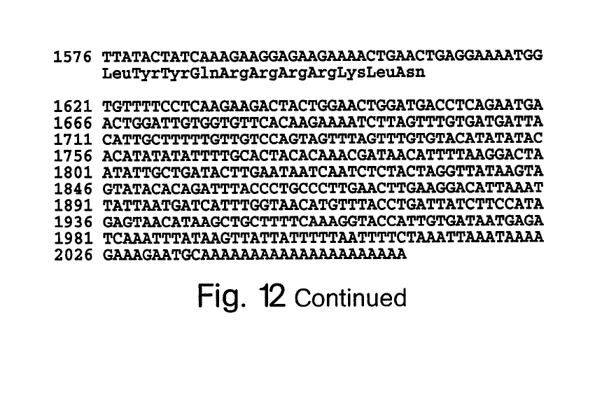

A SEC12 nucleic acid according to the invention includes the nucleic acid sequence (SEQ ID NO:22) of 20936375.0.104. The sequence is shown in FIG. 12. The disclosed nucleotide sequence is 1930 basepairs and includes an open reading frame (ORF) from nucleotides 7-1609. The encoded polypeptide includes 534 amino acid residues (SEQ ID NO:24) with a predicted molecular weight of 60037.3 Daltons. The encoded polypeptide is shown in FIG. 12 and is predicted by the PSORT computer program to be localized to the Plasma Membrane with a certainty of 0.7000. The SignalP computer program predicts that there is no cleavable N-terminal Signal Sequence, although the most likely cleavage site would appear to reside between residues 15 and 16 in the sequence SWC-CC.

The SEC12 protein has 453 of 531 residues (85%) identical to, and 482 of 531 residues (90%) positive with, a bovine brain membrane protein with activity as a diazepam receptor or agonist SWISSPROT-ACC:P07106, which is described in WO8604239. In view of the origin of this clone from kidney, as well as the prediction that it is localized in the plasma membrane, it is likely that a SEC12 polypeptide of the invention represents a receptor implicated in signaling pathways.

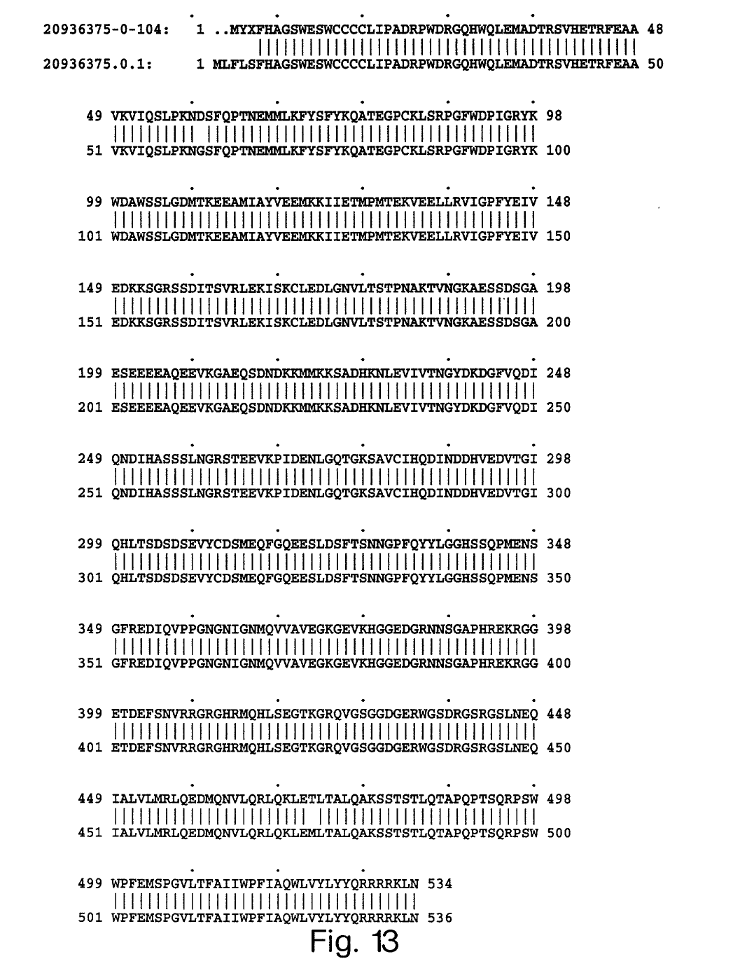

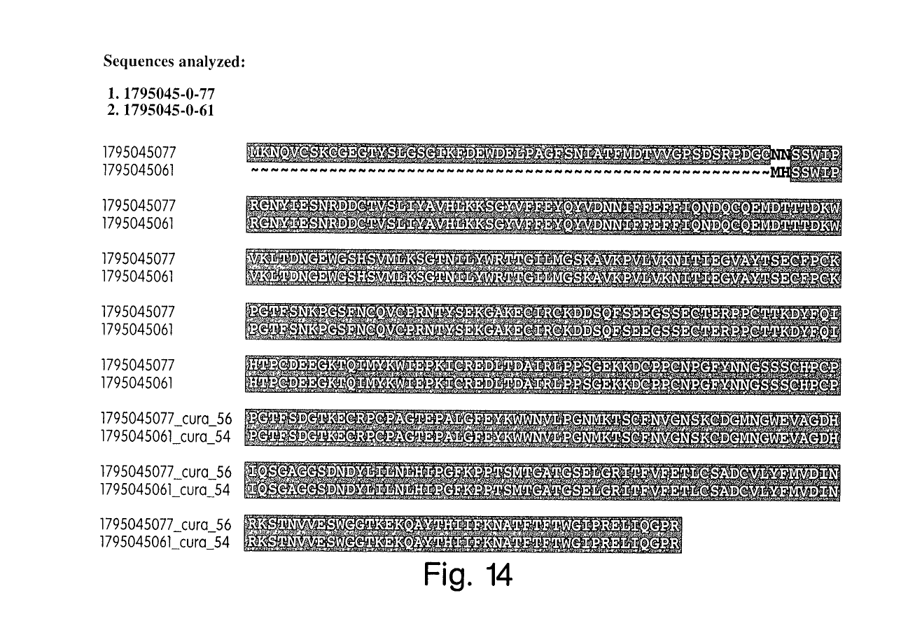

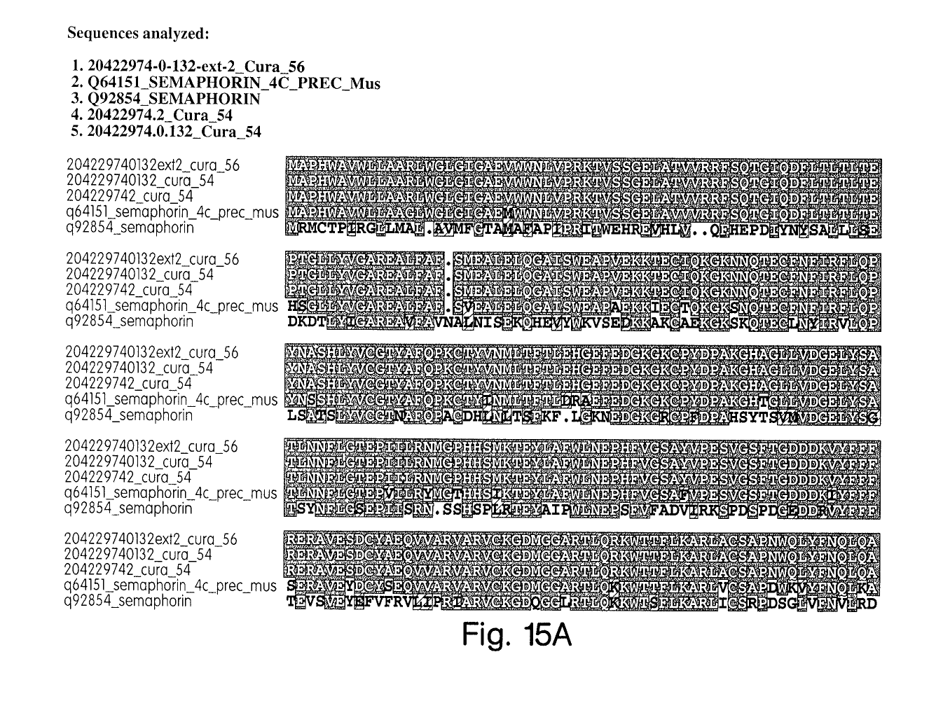

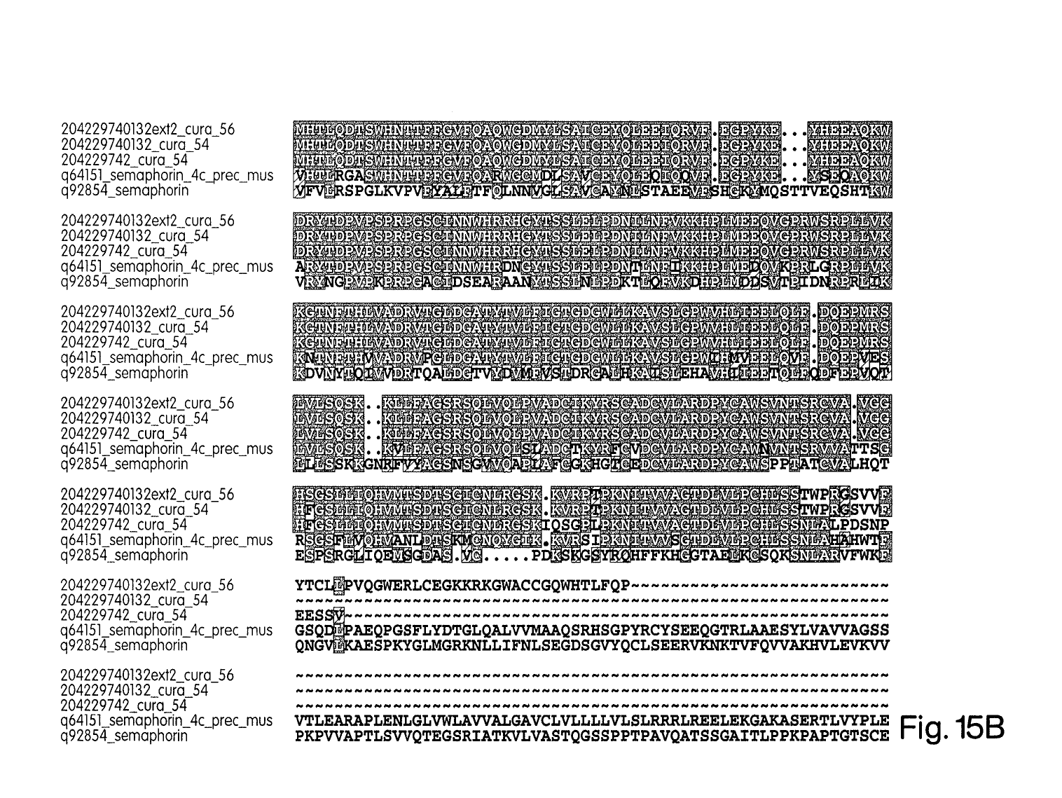

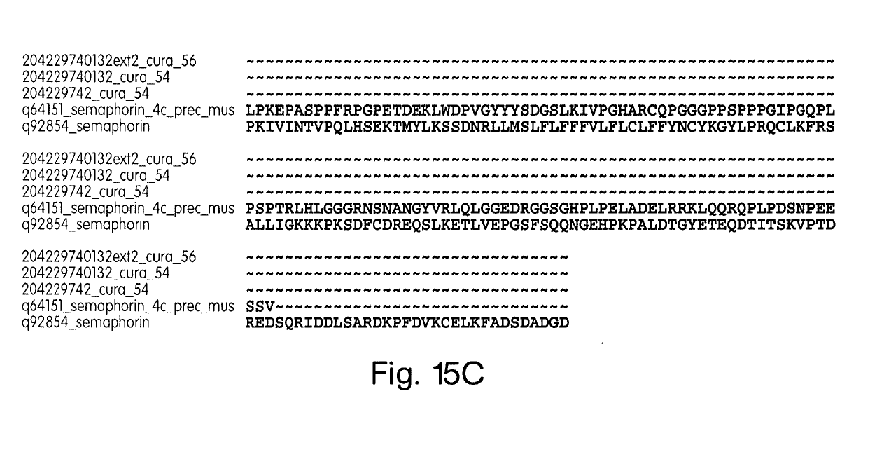

The disclosed SEC12 protein is related to the disclosed SEC8 polypeptide. An alignment of the SEC8 protein with the related SEC12 protein (SEQ ID NO:24) is illustrated in FIG. 13 and shows that these sequences are virtually identical, except for a mismatch at respective positions 472/474, and at the amino-terminal end.

The proteins of the invention encoded by SEC12 include the protein disclosed as being encoded by the ORF described herein, as well as any mature protein arising therefrom as a result of post-translational modifications. Thus, the proteins of the invention encompass both a precursor and any active forms of the SEC 12 protein.

### SEC Nucleic Acids

The novel nucleic acids of the invention include those that encode a SECX or SEC-like protein, or biologically-active portions thereof. The encoded polypeptides can thus include, e.g., the amino acid sequences of SEQ ID NO: 2, 4, 6, 8, 10, 12, 14, 16, 18, 20, 22, and/or 24.

In some embodiments, a SECX nucleic acid according to the invention encodes a mature form of a SECX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

In some embodiments, a nucleic acid encoding a polypeptide having the amino acid sequence of one or more of a SECX polypeptide includes the nucleic acid sequence of any of SEQ ID NOs:1, 3, 5, 7, 9, 11, 13, 15, 17, 19, 21, or 23, or a fragment thereof. Additionally, the invention includes mutant or variant nucleic acids of any of SEQ ID NOs:1, 3, 5, 7, 9, 11, 13, 15, 17, 19, 21, or 23, or a fragment thereof, any of whose bases may be changed from the disclosed sequence while still encoding a protein that maintains its SEC-like biological activities and physiological functions. The invention further includes the complement of the nucleic acid sequence of a SECX nucleic acid, e.g., SEQ ID NOs:1, 3, 5, 7, 9, 11, 13, 15, 17, 19, 21, 23, including fragments, derivatives, analogs and homologs thereof. The invention additionally includes nucleic acids or nucleic acid fragments, or complements thereto, whose structures include chemical modifications.

Also included are nucleic acid fragments sufficient for use as hybridization probes to identify SEC-encoding nucleic acids (e.g., SECX mRNA) and fragments for use as polymerase chain reaction (PCR) primers for the amplification or mutation of SECX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments, and homologs thereof. The nucleic acid molecule can be single-stranded or double-stranded, but preferably is double-stranded DNA.

The term “probes” refer to nucleic acid sequences of variable length, preferably between at least about 10 nucleotides (nt), 100 nt, or as many as about, e.g., 6,000 nt, depending upon the specific use. Probes are used in the detection of identical, similar, or complementary nucleic acid sequences. Longer length probes are usually obtained from a natural or recombinant source, are highly specific and much slower to hybridize than oligomers. Probes may be single- or double-stranded, and may also be designed to have specificity in PCR, membrane-based hybridization technologies, or ELISA-like technologies.

The term “isolated” nucleic acid molecule is a nucleic acid that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′-and 3′-termini of the nucleic acid) in the genomic DNA of the organism from which the nucleic acid is derived. For example, in various embodiments, the isolated SECX nucleic acid molecule can contain less than approximately 50 kb,25 kb,5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NOs: 1, 3, 5, 7, 9, 11, 13, 15, 17, 19, 21, or 23, or a complement of any of these nucleotide sequences, can be isolated using standard molecular biology techniques and the sequence information provided herein. Using all or a portion of the nucleic acid sequence of any of SEQ ID NOs: 1, 3, 5, 7, 9, 11, 13, 15, 17, 19, 21, or 23 as a hybridization probe, SECX nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to SECX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of any of SEQ ID NOs: 1, 3, 5, 7, 9, 11, 13, 15, 17, 19, 21, or 23, or a complement thereof. Oligonucleotides may be chemically synthesized and may also be used as probes.

In another embodiment, an isolated nucleic acid molecule of the invention includes a nucleic acid molecule that is a complement of the nucleotide sequence shown in any of SEQ ID NOs: 1, 3, 5, 7, 9, 11, 13, 15, 17, 19, 21, or 23. In still another embodiment, an isolated nucleic acid molecule of the invention includes a nucleic acid molecule that is a complement of the nucleotide sequence shown in any of SEQ ID NOs: 1, 3, 5, 7, 9, 11, 13, 15, 17, 19, 21, or 23, or a portion of this nucleotide sequence. A nucleic acid molecule that is complementary to the nucleotide sequence shown in is one that is sufficiently complementary to the nucleotide sequence shown in of any of SEQ ID NOs: 1, 3, 5, 7, 9, 11, 13, 15, 17, 19, 21, or 23 that it can hydrogen bond with little or no mismatches to the nucleotide sequence shown in of any of SEQ ID NOs: 1, 3, 5, 7, 9, 11, 13, 15, 17, 19, 21, or 23, thereby forming a stable duplex.

As used herein, the term “complementary” refers to Watson-Crick or Hoogsteen base- pairing between nucleotides units of a nucleic acid molecule, whereas the term “binding” is defined as the physical or chemical interaction between two polypeptides or compounds or associated polypeptides or compounds or combinations thereof. Binding includes ionic, non-ionic, Von der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Additionally, the nucleic acid molecule of the invention can comprise only a portion of the nucleic acid sequence of any of SEQ ID NOs: 1, 3, 5, 7, 9, 11, 13, 15, 17, 19, 21, or 23, e.g., a fragment that can be used as a probe or primer, or a fragment encoding a biologically active portion of a SECX polypeptide. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild-type.

Derivatives and analogs may be full-length or other than full-length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489), which is incorporated herein by reference in its entirety.

The term “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of SECX polypeptide. Isoforms can be expressed in different tissues of the same organism as a result of, e.g., alternative splicing of RNA. Alternatively, isoforms can be encoded by different genes. In the invention, homologous nucleotide sequences include nucleotide sequences encoding for a SECX polypeptide of species other than humans, including, but not limited to, mammals, and thus can include, e.g., mouse, rat, rabbit, dog, cat cow, horse, and other organisms. Homologous nucleotide sequences also include, but are not limited to, naturally-occurring allelic variations and mutations of the nucleotide sequences set forth herein. A homologous nucleotide sequence does not, however, include the nucleotide sequence encoding human SECX protein. Homologous nucleic acid sequences include those nucleic acid sequences that encode conservative amino acid substitutions (see below) in any of SEQ ID NOs: 2, 4, 6, 8, 10, 12, 14, 16, 18, 20, 22, or 24, as well as a polypeptide having SECX activity. Biological activities of the SECX proteins are described below. A homologous amino acid sequence does not encode the amino acid sequence of a human SECX polypeptide.

The nucleotide sequence determined from the cloning of the human SECX gene allows for the generation of probes and primers designed for use in identifying the cell types disclosed and/or cloning SECX homologues in other cell types, e.g., from other tissues, as well as SECX homologues from other mammals. The probe/primer typically includesa substantially-purified oligonucleotide. The oligonucleotide typically includesa region of nucleotide sequence that hybridizes under stringent conditions to at least about 12, 25, 50, 100, 150, 200, 250, 300, 350 or 400 or more consecutive sense strand nucleotide sequence of a SECX nucleic acid, e.g., one including all or a portion of any of SEQ ID NOs: 1, 3, 5, 7, 9, 11, 13, 15, 17, 19, 21, or 23. Alternatively, the oligonucleotide sequence may include a region of nucleotide sequences that hybridizes to some or all of an anti-sense strand of a strand encoding a SECX nucleic acid. For example, the oligonucleotide may include some or all of the anti-sense strand nucleotide sequence of SEQ ID NOs: 1, 3, 5, 7, 9, 11, 13, 15, 17, 19, 21, or 23 or of a naturally occurring mutant of one of these nucleic acids.

Probes based upon the human SECX nucleotide sequence can be used to detect transcripts or genomic sequences encoding the same or homologous proteins. In various embodiments, the probe further includesa label group attached thereto, e.g., the label group can be a radioisotope, a fluorescent compound, an enzyme, or an enzyme co-factor. Such probes can be used as a part of a diagnostic test kit for identifying cells or tissue which mis-express a SECX protein, such as by measuring a level of a SECX-encoding nucleic acid in a sample of cells from a subject e.g., detecting SECX mRNA levels or determining whether a genomic SECX gene has been mutated or deleted.

The term “a polypeptide having a biologically-active portion of SECX” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of SECX” can be prepared by isolating a portion of a nucleotide, e.g., a nucleotide including a portion of SEQ ID NO:1, 3, 5, 7, 9, 11.13, 15, 17, 19, 21, or 23, that encodes a polypeptide having a SECX biological activity (biological activities of the SECX proteins are summarized in TABLE 1), expressing the encoded portion of SECX protein (e.g., by recombinant expression in vitro) and assessing the activity of the encoded portion of SECX.

### SECX Variants

The invention further encompasses nucleic acid molecules that differ from the disclosed SECX nucleotide sequences due to degeneracy of the genetic code. These nucleic acids can encode the same SECX protein as those encoded by the nucleotide sequence shown in SEQ ID NO:1, 3, 5, 7, 9, 11, 13, 15, 17, 19, 21, or 23. In another embodiment, an isolated nucleic acid molecule of the invention has a nucleotide sequence encoding a protein having an amino acid sequence shown in any of SEQ ID NO:2, 4, 6, 8, 10, 12, 14, 16, 18, 20, 22, or 24.

In addition to the human SECX nucleotide sequence shown in any of SEQ ID NO:1, 3, 5, 7, 9, 11, 13, 15, 17, 19, 21, or 23, it will be appreciated by those skilled in the art that DNA sequence polymorphisms that lead to changes in the amino acid sequences of SECX may exist within a population (e.g., the human population). Such genetic polymorphism in the SECX gene may exist among individuals within a population due to natural allelic variation. As used herein, the terms “gene” and “recombinant gene” refer to nucleic acid molecules comprising an open reading frame encoding a SECX protein, preferably a mammalian SECX protein. Such natural allelic variations can typically result in 1-5% variance in the nucleotide sequence of the SECX gene. Any and all such nucleotide variations and resulting amino acid polymorphisms in SECX that are the result of natural allelic variation and that do not alter the functional activity of SECX are intended to be within the scope of the invention.

Additionally, nucleic acid molecules encoding SECX proteins from other species, and thus that have a nucleotide sequence that differs from the nucleic acid sequence of a human SECX nucleic acid (e.g., it differs from SEQ ID NO:1, 3, 5, 7, 9, 11, 13, 15, 17, 19, 21, or 23), are intended to be within the scope of the invention. Nucleic acid molecules corresponding to natural allelic variants and homologues of the SECX cDNAs of the invention can be isolated based on their homology to the human SECX nucleic acids disclosed herein using the human cDNAs, or a portion thereof, as a hybridization probe according to standard hybridization techniques under stringent hybridization conditions.

In another embodiment, an isolated nucleic acid molecule of the invention is at least 6 nucleotides in length and hybridizes under stringent conditions to the nucleic acid molecule comprising the nucleotide sequence of a SEC X nucleic acid, e.g., SEQ ID NO:1, 3, 5, 7, 9, 11, 13, 15, 17, 19, 21, or 23. In another embodiment, the nucleic acid is at least 10, 25, 50, 100, 250, 500 or 750 nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding SECX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tₘ) for the specific sequence at a defined ionic strength and pH. The Tₘ is the temperature (under defined ionic strength, pH and nucleic acid concentration) at which 50% of the probes complementary to the target sequence hybridize to the target sequence at equilibrium. Since the target sequences are generally present at excess, at Tₘ, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6+SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of a SECX nucleic acid, including those described herein, corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of a SECX nucleic acid (e.g., SEQ ID NO:1, 3, 5, 7, 9, 11, 13, 15, 17, 19, 21, or 23), or fragments, analogs or derivatives thereof, under conditions of moderate stringency is provided. A non-limiting example of moderate stringency hybridization conditions are hybridization in 6X SSC, 5X Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, New York, and Kriegler, 1990. GENE TRANSFER AND EXPRESSION, A LABORATORY MANUAL, Stockton Press, New York.

In a third embodiment, a nucleic acid that is hybridizable to the nucleic acid molecule comprising the nucleotide sequence of any SECX nucleic acid (e.g., it hybridizes to SEQ ID NO:1, 3, 5, 7, 9, 11, 13, 15, 17, 19, 21, or 23), or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA. 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-SECXecies hybridizations). See, e.g., Ausubel, et al., (eds.), 1993. CURRENT PROTOCOLS IN MOLECULAR BIOLOGY, John Wiley & Sons, New York, and Kriegler, 1990. GENE TRANSFER AND EXPRESSION, A LABORATORY MANUAL, Stockton Press, New York; Shilo and Weinberg, 1981. Proc. Natl. Acad. Sci. USA 78: 6789-6792.

### Conservative Mutations

In addition to naturally-occurring allelic variants of the SECX sequence that may exist in the population, the skilled artisan will further appreciate that changes can be introduced by mutation into the nucleotide sequence of a SECX nucleic acid (e.g., SEQ ID NO:1, 3, 5, 7, 9, 11, 13, 15, 17, 19, 21, or 23), thereby leading to changes in the amino acid sequence of the encoded SECX protein, without altering the functional ability of the SECX protein. For example, nucleotide substitutions leading to amino acid substitutions at “non-essential” amino acid residues can be made in the sequence of any of SEQ ID NO:1, 3, 5, 7, 9, 11, 13, 15, 17, 19, 21, or 23. A “non-essential” amino acid residue is a residue that can be altered from the wild-type sequence of SECX without altering the biological activity, whereas an “essential” amino acid residue is required for biological activity. For example, amino acid residues that are conserved among the SECX proteins of the invention, are predicted to be particularly non-amenable to such alteration.

Amino acid residues that are conserved among members of a SECX family members are predicted to be less amenable to alteration. For example, a SECX protein according to the invention can contain at least one domain (e.g., as shown in TABLE 1) that is a typically conserved region in a SECX family member. As such, these conserved domains are not likely to be amenable to mutation. Other amino acid residues, however, (e.g., those that are not conserved or only semi-conserved among members of the SECX family) may not be as essential for activity and thus are more likely to be amenable to alteration.

Another aspect of the invention pertains to nucleic acid molecules encoding SECX proteins that contain changes in amino acid residues that are not essential for activity. Such SECX proteins differ in amino acid sequence from the amino acid sequence of an SECX polypeptide (e.g., SEQ ID NO:2, 4, 6, 8, 10, 12, 14, 16, 18, 20, 22, or 24), yet retain biological activity. In one embodiment, the isolated nucleic acid molecule includesa nucleotide sequence encoding a protein, wherein the protein includes an amino acid sequence at least about 75% homologous to the amino acid sequence of any of SEQ ID NO:2, 4, 6, 8, 10, 12, 14, 16, 18, 20, 22, or 24. Preferably, the protein encoded by the nucleic acid is at least about 80% homologous to any of SEQ ID NO:2, 4, 6, 8, 10, 12, 14, 16, 18, 20, 22, or 24, more preferably at least about 90%, 95%, 98%, and most preferably at least about 99% homologous to SEQ ID NO:2, 4, 6, 8, 10, 12, 14, 16, 18, 20, 22, or 24.

An isolated nucleic acid molecule encoding a SECX protein homologous to a SECX poloypeptide, e.g. a polypeptide including the amino acid sequence of any of SEQ ID NO:2, 4, 6, 8, 10, 12, 14, 16, 18, 20, 22, or 24, can be created by introducing one or more nucleotide substitutions, additions or deletions into the corresponding SECX nucleotide sequence, such that one or more amino acid substitutions, additions or deletions are introduced into the encoded protein.

Mutations can be introduced into SECX nucleic acid by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Thus, a predicted nonessential amino acid residue in SECX is replaced with another amino acid residue from the same side chain family. Alternatively, in another embodiment, mutations can be introduced randomly along all or part of a SECX coding sequence, such as by saturation mutagenesis, and the resultant mutants can be screened for SECX biological activity to identify mutants that retain activity. Following mutagenesis of the SECX nucleic acid, the encoded protein can be expressed by any recombinant technology known in the art and the activity of the protein can be determined.

In one embodiment, a mutant SECX protein can be assayed for: (i) the ability to form protein:protein interactions with other SECX proteins, other cell-surface proteins, or biologically-active portions thereof; (ii) complex formation between a mutant SECX protein and a SECX receptor; (iii) the ability of a mutant SECX protein to bind to an intracellular target protein or biologically active portion thereof; (e.g., avidin proteins); (iv) the ability to bind BRA protein; or (v) the ability to specifically bind an anti-SECX protein antibody.

### Antisense Nucleic Acids

Another aspect of the invention pertains to isolated antisense nucleic acid molecules that are hybridizable to or complementary to the nucleic acid molecule including a SECX nucleic acid (e.g. a nucleic acid including SEQ ID NOs: 1, 3, 5, 7, 9, 11, 13, 15, 17, 19, 21 or 23), or fragments, analogs or derivatives thereof. An “antisense” nucleic acid includesa nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire SECX coding strand, or to only a portion thereof.

In one embodiment, an antisense nucleic acid molecule is antisense to a “coding region” of the coding strand of a nucleotide sequence encoding SECX. The term “coding region” refers to the region of the nucleotide sequence comprising codons which are translated into amino acid residues (e.g., the protein coding region of SEQ ID NO: 1, 3, 5, 7, 9, 11, 13, 15, 17, 19, 21, or 23). In another embodiment, the antisense nucleic acid molecule is antisense to a “non-coding region” of the coding strand of a SECX nucleotide sequence. The term “non-coding region” refers to 5′ and 3′ sequences which flank the coding region that are not translated into amino acids (i.e., also referred to as 5′ and 3′ non-translated regions).

Given the coding strand sequences encoding SECX disclosed herein, antisense nucleic acids of the invention can be designed according to the rules of Watson and Crick or Hoogsteen base-pairing. The antisense nucleic acid molecule can be complementary to the entire coding region of SECX mRNA, but more preferably is an oligonucleotide that is antisense to only a portion of the coding or non-coding region of SECX mRNA. For example, the antisense oligonucleotide can be complementary to the region surrounding the translation start site of SECX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids, e.g., phosphorothioate derivatives and acridine-substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a SECX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual α-units, the strands run parallel to each other (Gaultier, et al., 1987. Nucl. Acids Res. 15: 6625-6641). The antisense nucleic acid molecule can also comprise a 2′-o-methylribonucleotide (Inoue, et al., 1987. Nucl. Acids Res. 15: 6131-6148) or a chimeric RNA-DNA analogue (Inoue, et al., 1987. FEBS Lett. 215: 327-330).

### Ribozymes and PNA Moieties

Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes; described by Haselhoff and Gerlach, 1988. Nature 334: 585-591) can be used to catalytically-cleave SECX mRNA transcripts to thereby inhibit translation of SECX mRNA. A ribozyme having specificity for a SECX nucleic acid can be designed based upon the nucleotide sequence of a SECX DNA disclosed herein (e.g., SEQ ID NO:1,3, 5, 7, 9, 11, 13, 15, 17, 19, 21, or 23). For example, a derivative of a Tetrahymena L-19 IVS RNA can be constructed in which the nucleotide sequence of the active site is complementary to the nucleotide sequence to be cleaved in a SECX-encoding mRNA. See, e.g., Cech, et al., U.S. Pat. No. 4,987,071; and Cech, et al., U.S. Pat. No. 5,116,742. Alternatively, SECX mRNA can be used to select a catalytic RNA having a specific ribonuclease activity from a pool of RNA molecules (Bartel, et al., 1993. Science 261: 1411-1418).

Alternatively, SECX gene expression can be inhibited by targeting nucleotide sequences complementary to the regulatory region of the SECX (e.g., the SECX promoter and/or enhancers) to form triple helical structures that prevent transcription of the SECX gene in target cells. See, e.g., Helene, 1991. Anticancer Drug Des. 6: 569-84; Helene, et al., 1992. Ann. N.Y. Acad. Sci. 660: 27-36; and Maher, 1992. Bioassays 14: 807-15.

In various embodiments, the nucleic acids of SECX can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (Hyrup, et al., 1996. Bioorg. Med. Chem. 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. above; Perry-O'Keefe, et al., 1996. Proc. Natl. Acad. Sci. USA 93: 14670-14675.

PNAs of SECX can be used in therapeutic and diagnostic applications. For example, PNAs can be used as antisense or antigene agents for sequence-SECXecific modulation of gene expression by, e.g., inducing transcription or translation arrest or inhibiting replication. PNAs of SECX can also be used, e.g., in the analysis of single base pair mutations in a gene by, e.g., PNA directed PCR clamping; as artificial restriction enzymes when used in combination with other enzymes, e.g., S1 nucleases (see, Hyrup, 1996., above); or as probes or primers for DNA sequence and hybridization (see, Hyrup, et al., 1996.; Perry-O'Keefe, 1996., above).

In another embodiment, PNAs of SECX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of SECX can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, 1996., above). The synthesis of PNA-DNA chimeras can be performed as described in Finn, et al., (1996. Nucl. Acids Res. 24: 3357-3363). For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag, et al., 1989. Nucl. Acid Res. 17: 5973-5988). PNA monomers are then coupled in a stepwise manner to produce a chimeric molecule with a 5′ PNA segment and a 3′ DNA segment (see, Finn, et al., 1996., above). Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

### SECX Polypeptides

A polypeptide according to the invention includes a polypeptide including the amino acid sequence of SECX polypeptides. In some embodiments, the SECX polypeptide includes the amino acid sequence of SEQ ID NO:2, 4, 6, 8, 10, 12, 14, 16, 18, 20, 22, or 24. In various embodiments, a SECX polypeptide is provided in a form longer than the sequence of the mature SECX polypeptide. For example, a SECX polypeptide may be provided as including an amino terminal signal sequence. In other embodiments, the SECX polypeptide is provided as the mature form of the polypeptide.

The invention also includes a mutant or variant protein any of whose residues may be changed from the corresponding residues shown in SEQ ID NO:2, 4, 6, 8, 10, 12, 14, 16, 18, 20, 22, or 24, while still encoding a protein that maintains its SECX activities and physiological functions, or a functional fragment thereof.

In general, a SECX variant that preserves SECX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated SECX proteins, and biologically-active portions thereof, or derivatives, fragments, analogs or homologs thereof. Also provided are polypeptide fragments suitable for use as immunogens to raise anti-SECX antibodies. In one embodiment, native SECX proteins can be isolated from cells or tissue sources by an appropriate purification scheme using standard protein purification techniques. In another embodiment, SECX proteins are produced by recombinant DNA techniques. Alternative to recombinant expression, a SECX protein or polypeptide can be synthesized chemically using standard peptide synthesis techniques.

An “purified” polypeptide or protein or biologically-active portion thereof is substantially free of cellular material or other contaminating proteins from the cell or tissue source from which the SECX protein is derived, or substantially free from chemical precursors or other chemicals when chemically synthesized. The language “substantially free of cellular material” includes preparations of SECX proteins in which the protein is separated from cellular components of the cells from which it is isolated or recombinantly-produced. In one embodiment, the language “substantially free of cellular material” includes preparations of SECX proteins having less than about 30% (by dry weight) of a non-SECX protein (also referred to herein as a “contaminating protein”), more preferably less than about 20% of a non-SECX protein, still more preferably less than about 10% of a non-SECX protein, and most preferably less than about 5% of a non-SECX protein. When the SECX protein or biologically-active portion thereof is recombinantly-produced, it is also preferably substantially free of culture medium, i.e., culture medium represents less than about 20%, more preferably less than about 10%, and most preferably less than about 5% of the volume of the SECX protein preparation.

The phrase “substantially free of chemical precursors or other chemicals” includes preparations of SECX protein in which the protein is separated from chemical precursors or other chemicals that are involved in the synthesis of the protein. In one embodiment, the language “substantially free of chemical precursors or other chemicals” includes preparations of SECX protein having less than about 30% (by dry weight) of chemical precursors or non-SECX chemicals, more preferably less than about 20% chemical precursors or non-SECX chemicals, still more preferably less than about 10% chemical precursors or non-SECX chemicals, and most preferably less than about 5% chemical precursors or non-SECX chemicals.

Biologically-active portions of a SECX protein include peptides comprising amino acid sequences sufficiently homologous to or derived from the amino acid sequence of the SECX protein which include fewer amino acids than the full-length SECX proteins, and exhibit at least one activity of a SECX protein. Typically, biologically-active portions comprise a domain or motif with at least one activity of the SECX protein. A biologically-active portion of a SECX protein can be a polypeptide which is, for example, 10, 25, 50, 100 or more amino acids in length.

A biologically-active portion of a SECX protein of the invention may contain at least one of the above-identified conserved domains. Moreover, other biologically active portions, in which other regions of the protein are deleted, can be prepared by recombinant techniques and evaluated for one or more of the functional activities of a native SECX protein.

In some embodiments, the SECX protein is substantially homologous to any of SEQ ID NO:2, 4, 6, 8, 10, 12, 14, 16, 18, 20, 22, or 24, and retains the functional activity of the protein of any of the SECX protein, yet differs in amino acid sequence due to natural allelic variation or mutagenesis, as described in detail below. Accordingly, in another embodiment, the SECX protein is a protein that includesan amino acid sequence at least about 45% homologous, and more preferably about 55, 65, 70, 75, 80, 85, 90, 95, 98 or even 99% homologous to the amino acid sequence of any of SEQ ID NO:2, 4, 6, 8, 10, 12, 14, 16, 18, 20, 22, or 24 and retains the functional activity of the corresponding SECX proteins of the corresponding polypeptide having the sequence of SEQ ID NO:2, 4, 6, 8, 10, 12, 14, 16, 18, 20, 22, or 24.

### Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J. Mol. Biol. 48: 443453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOs: 1, 3, 5, 7, 9, 11, 13, 15, 17, 19, 21, or 23.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide includesa sequence that has at least 80 percent sequence identity, preferably at least 85 percent identity and often 90 to 95 percent sequence identity, more usually at least 99 percent sequence identity as compared to a reference sequence over a comparison region.

### Chimeric and Fusion Proteins

The invention also provides SECX chimeric or fusion proteins. As used herein, a SECX “chimeric protein” or “fusion protein” includesa SECX polypeptide operatively-linked to a non-SECX polypeptide. An “SECX polypeptide” refers to a polypeptide having an amino acid sequence corresponding to a SECX protein shown in, e.g., SEQ ID NO:2, 4, 6, 8, 10, 12, 14, 16, 18, 20, 22, and/or 24. A “non-SECX polypeptide” or “non-SECX protein” refers to a polypeptide having an amino acid sequence corresponding to a protein that is not substantially homologous to a SECX polypeptide (e.g., a protein that is different from the SECX protein and that is derived from the same or a different organism). Within a SECX fusion protein the SECX polypeptide can correspond to all or a portion of a SECX protein. In one embodiment, a SECX fusion protein includes at least one biologically-active portion of a SECX protein. In another embodiment, a SECX fusion protein comprises at least two biologically-active portions of a SECX protein. In yet another embodiment, a SECX fusion protein comprises at least three biologically-active portions of a SECX protein. Within the fusion protein, the term “operatively-linked” is intended to indicate that the SECX polypeptide and the non-SECX polypeptide are fused in-frame with one another. The non-SECX polypeptide can be fused to the amino-terminus or carboxyl-terminus of the SECX polypeptide.

In one embodiment, the fusion protein is a GST-SECX fusion protein in which the SECX sequences are fused to the carboxyl-terminus of the GST (glutathione S-transferase) sequences. Such fusion proteins can facilitate the purification of recombinant SECX polypeptides.

In another embodiment, the fusion protein is a SECX protein containing a heterologous signal sequence at its amino-terminus. In certain host cells (e.g., mammalian host cells), expression and/or secretion of SECX can be increased through use of a heterologous signal sequence.

In yet another embodiment, the fusion protein is a SECX-immunoglobulin fusion protein in which the SECX sequences are fused to sequences derived from a member of the immunoglobulin protein family. The SECX-immunoglobulin fusion proteins of the invention can be incorporated into pharmaceutical compositions and administered to a subject to inhibit an interaction between a SECX ligand and a SECX protein on the surface of a cell, to thereby suppress SECX-mediated signal transduction in vivo. The SECX-immunoglobulin fusion proteins can be used to affect the bioavailability of a SECX cognate ligand. Inhibition of the SECX ligand/interaction may be useful therapeutically for both the treatment of proliferative and differentiative disorders, as well as modulating (e.g., promoting or inhibiting) cell survival. Moreover, the SECX-immunoglobulin fusion proteins of the invention can be used as immunogens to produce anti-SECX antibodies in a subject, to purify SECX ligands, and in screening assays to identify molecules that inhibit the interaction of SECX with a SECX ligand.

A SECX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and re-amplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A SECX-encoding nucleic acid can be cloned into such an expression vector such that the fusion moiety is linked in-frame to the SECX protein.

### SECX Agonists and Antagonists

The invention also pertains to variants of the SECX proteins that function as either SECX agonists (i.e., mimetics) or as SECX antagonists. Variants of the SECX protein can be generated by mutagenesis (e.g., discrete point mutation or truncation of the SECX protein). An agonist of a SECX protein can retain substantially the same, or a subset of, the biological activities of the naturally-occurring form of a SECX protein. An antagonist of a SECX protein can inhibit one or more of the activities of the naturally occurring form of a SECX protein by, for example, competitively binding to a downstream or upstream member of a cellular signaling cascade which includes the SECX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the SECX proteins.

Variants of the SECX proteins that function as either SECX agonists (i.e., mimetics) or as SECX antagonists can be identified by screening combinatorial libraries of mutants (e.g., truncation mutants) of the SECX proteins for SECX protein agonist or antagonist activity. In one embodiment, a variegated library of SECX variants is generated by combinatorial mutagenesis at the nucleic acid level and is encoded by a variegated gene library. A variegated library of SECX variants can be produced by, for example, enzymatically-ligating a mixture of synthetic oligonucleotides into gene sequences such that a degenerate set of potential SECX sequences is expressible as individual polypeptides, or alternatively, as a set of larger fusion proteins (e.g., for phage display) containing the set of SECX sequences therein. There are a variety of methods which can be used to produce libraries of potential SECX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential SECX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

### Polypeptide Libraries

In addition, libraries of fragments of the SECX protein coding sequences can be used to generate a variegated population of SECX fragments for screening and subsequent selection of variants of a SECX protein. In one embodiment, a library of coding sequence fragments can be generated by treating a double-stranded PCR fragment of a SECX coding sequence with a nuclease under conditions wherein nicking occurs only about once per molecule, denaturing the double stranded DNA, renaturing the DNA to form double-stranded DNA that can include senselantisense pairs from different nicked products, removing single stranded portions from reformed duplexes by treatment with S₁ nuclease, and ligating the resulting fragment library into an expression vector. By this method, expression libraries can be derived which encodes amino-terminal and internal fragments of various sizes of the SECX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of SECX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify SECX variants. See, e.g., Arkin and Yourvan, 1992. Proc. Natl. Acad. Sci. USA 89: 7811-7815; Delgrave, et al., 1993. Protein Engineering 6:327-331.

### Anti-SECX Antibodies

The invention encompasses antibodies and antibody fragments, such as F_{ab} or (F_{ab})₂. that bind immunospecifically to any of the SECX polypeptides of said invention.

An isolated SECX protein, or a portion or fragment thereof can be used as an immunogen to generate antibodies that bind to SECX polypeptides using standard techniques for polyclonal and monoclonal antibody preparation. The full-length SECX proteins can be used or, alternatively, the invention provides antigenic peptide fragments of SECX proteins for use as immunogens. The antigenic SECX peptides comprises at least 4 amino acid residues of an SECX polypeptide, e.g., the amino acid sequence of SEQ ID NO:2, 4, 6, 8, 10, 12, 14, 16, 18, 20, 22, or 24 and encompasses an epitope of SECX such that an antibody raised against the peptide forms a specific immune complex with SECX. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of SECX that is located on the surface of the protein (e.g., a hydrophilic region). As a means for targeting antibody production, hydropathy plots showing regions of hydrophilicity and hydrophobicity may be generated by any method well known in the art, including, for example, the Kyte-Doolittle or the Hopp-Woods methods, either with or without Fourier transformation (see, e.g., Hopp and Woods, 1981. Proc. Nat. Acad. Sci. USA 78: 3824-3828; Kyte and Doolittle, 1982. J. Mol. Biol. 157: 105-142, each incorporated herein by reference in their entirety).

SECX protein sequences including, e.g., SEQ ID NO:2, 4, 6, 8, 10, 12, 14, 16, 18, 20, 22, or 24) or derivatives, fragments, analogs, or homologs thereof, may be used as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (i.e., immunoreacts with) an antigen, such as SECX. Such antibodies include, but are not limited to, polyclonal, monoclonal, chimeric, single chain, F_{ab} and F_{(ab′)2} fragments, and an F_{ab} expression library. In a specific embodiment, antibodies to human SECX proteins are disclosed. Various procedures known within the art may be used for the production of polyclonal or monoclonal antibodies to a SECX protein sequence, e.g., a protein sequence of SEQ ID NO:2, 4, 6, 8, 10, 12, 14, 16, 18, 20, 22, or 24, or a derivative, fragment, analog, or homolog thereof.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed SECX protein or a chemically-synthesized SECX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against SECX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of SECX. A monoclonal antibody composition thus typically displays a single binding affinity for a particular SECX protein with which it immunoreacts. For preparation of monoclonal antibodies directed towards a particular SECX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp.77-96). Each of the above citations is incorporated herein by reference in their entirety.

According to the invention, techniques can be adapted for the production of single-chain antibodies specific to a SECX protein (see, e.g., U.S. Pat. No. 4,946,778). In addition, methods can be adapted for the construction of F_{ab} expression libraries (see, e.g., Huse, et al., 1989. Science 246: 1275-1281) to allow rapid and effective identification of monoclonal F_{ab} fragments with the desired specificity for a SECX protein or derivatives, fragments, analogs or homologs thereof. Non-human antibodies can be “humanized” by techniques well-known within the art. See, e.g., U.S. Pat. No. 5,225,539. Antibody fragments that contain the idiotypes to a SECX protein may be produced by techniques known in the art including, but not limited to: (i) an F_{(ab′)}₂ fragment produced by pepsin digestion of an antibody molecule; (ii) an F_{ab} fragment generated by reducing the disulfide bridges of an F_{(ab′)}₂ fragment; (iii) an F_{ab} fragment generated by the treatment of the antibody molecule with papain and a reducing agent and (iv) Fᵥ fragments.

Additionally, recombinant anti-SECX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314 :446-449; Shaw, et al., 1988. J. Natl. Cancer Inst. 80: 1553-1559); Morrison(1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other inmmunologically-mediated techniques known within the art. In a specific embodiment, selection of antibodies that are specific to a particular domain of a SECX protein is facilitated by generation of hybridomas that bind to the fragment of a SECX protein possessing such a domain. Thus, antibodies that are specific for a desired domain within a SECX protein, or derivatives, fragments, analogs or homologs thereof, are also provided herein.

Anti-SECX antibodies may be used in methods known within the art relating to the localization and/or quantitation of a SECX protein (e.g., for use in measuring levels of the SECX protein within appropriate physiological samples, for use in diagnostic methods, for use in imaging the protein, and the like). In a given embodiment, antibodies for SECX proteins, or derivatives, fragments, analogs or homologs thereof, that contain the antibody derived binding domain, are utilized as pharmacologically-active compounds (hereinafter “Therapeutics”).

An anti-SECX antibody (e.g., monoclonal antibody) can be used to isolate a SECX polypeptide by standard techniques, such as affinity chromatography or immunoprecipitation. An anti-SECX antibody can facilitate the purification of natural SECX polypeptide from cells and of recombinantly-produced SECX polypeptide expressed in host cells. Moreover, an anti-SECX antibody can be used to detect SECX protein (e.g., in a cellular lysate or cell supernatant) in order to evaluate the abundance and pattern of expression of the SECX protein. Anti-SECX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### SECX Recombinant Expression Vectors and Host Cells

Another aspect of the invention pertains to vectors, preferably expression vectors, containing a nucleic acid encoding a SECX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably, as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The phrase “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-SECXecific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., SECX proteins, mutant forms of SECX proteins, fusion proteins, etc.).

The recombinant expression vectors of the invention can be designed for expression of SECX proteins in prokaryotic or eukaryotic cells. For example, SECX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T₇ promoter regulatory sequences and T₇ polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xₐ, thrombin, and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion Escherichia coli expression vectors include pTrc (Amrann et al., (1988) Gene 69:301-315) and pET 11d (Studier, et al., GENE EXPRESSION TECHNOLOGY: METHODS IN ENZYMOLOGY 185, Academic Press, San Diego, Calif. (1990) 60-89).

One strategy to maximize recombinant protein expression in Escherichia coli is to express the protein in a host bacteria with an impaired capacity to proteolytically-cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in Escherichia coli (see, e.g., Wada, et al., 1992. Nucl. Acids Res. 20: 2111-2118). Such alteration of nucleic acid sequences of the invention can be carried out by standard DNA synthesis techniques.

In another embodiment, the SECX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, SECX can be expressed in insect cells using baculovirus expression vectors. Baculovirus vectors available for expression of proteins in cultured insect cells (e g., SF9 cells) include the pAc series (Smith, et al., 1983. Mol. Cell. Biol 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-SECXecific regulatory elements are used to express the nucleic acid). Tissue-SECXecific regulatory elements are known in the art. Non-limiting examples of suitable tissue-SECXecific promoters include the albumin promoter (liver-SECXecific; see, Pinkert, et al., 1987. Genes Dev. 1: 268-277), lymphoid-SECXecific promoters (see, Calame and Eaton, 1988. Adv. Immunol. 43: 235-275), in particular promoters of T cell receptors (see, Winoto and Baltimore, 1989. EMBO J. 8: 729-733) and immunoglobulins (see, Banerji, et al., 1983. Cell 33: 729-740; Queen and Baltimore, 1983. Cell 33: 741-748), neuron-SECXecific promoters (e.g., the neurofilament promoter; see, Byrne and Ruddle, 1989. Proc. Natl. Acad. Sci. USA 86: 5473-5477), pancreas-SECXecific promoters (see, Edlund, et al., 1985. Science 230: 912-916), and mammary gland-SECXecific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (see, Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to SECX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et at., “Antisense RNA as a molecular tool for genetic analysis, ” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, SECX protein can be expressed in bacterial cells such as Escherichia coli, insect cells, yeast or mammalian cells (such as Chinese hamster ovary cells ((CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding SECX or can be introduced on a separate vector. Cells stably-transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) SECX protein. Accordingly, the invention further provides methods for producing SECX protein using the host cells of the invention. In one embodiment, the method comprises culturing the host cell of invention (i.e., into which a recombinant expression vector encoding SECX protein has been introduced) in a suitable medium such that SECX protein is produced. In another embodiment, the method further comprises isolating SECX protein from the medium or the host cell.

### Transgenic Animals

The host cells of the invention can also be used to produce non-human transgenic animals. For example, in one embodiment, a host cell of the invention is a fertilized oocyte or an embryonic stem cell into which SECX protein-coding sequences have been introduced. These host cells can then be used to create non-human transgenic animals in which exogenous SECX sequences have been introduced into their genome or homologous recombinant animals in which endogenous SECX sequences have been altered. Such animals are useful for studying the function and/or activity of SECX protein and for identifying and/or evaluating modulators of SECX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc.

A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous SECX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing SECX-encoding nucleic acid into the male pronuclei of a fertilized oocyte (e.g., by micro-injection, retroviral infection) and allowing the oocyte to develop in a pseudopregnant female foster animal. The human SECX DNA sequences, e.g., SEQ ID NOs: 1, 3, 5, 7, 9, 11, 13, 15, 17, 19, 21 or 34, can be introduced as a transgene into the genome of a non-human animal. Alternatively, a non-human homologue of a human SECX gene, such as a mouse SECX gene, can be isolated based on hybridization to a human SECX DNA and used as a transgene. Intronic sequences and polyadenylation signals can also be included in the transgene to increase the efficiency of expression of the transgene. A tissue-specific regulatory sequence(s) can be operably-linked to the SECX transgene to direct expression of SECX protein to particular cells. Methods for generating transgenic animals via embryo manipulation and micro-injection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the SECX transgene in its genome and/or expression of SECX mRNA in tissues or cells of the animals. A transgenic founder animal can then be used to breed additional animals carrying the transgene. Moreover, transgenic animals carrying a transgene-encoding SECX protein can further be bred to other transgenic animals carrying other transgenes.

To create a homologous recombinant animal, a vector is prepared which contains at least a portion of a SECX gene into which a deletion, addition or substitution has been introduced to thereby alter, e.g., functionally disrupt, the SECX gene. The SECX gene can be a human gene (e.g., SEQ ID NO: 1, 3, 5, 7, 9, 11, 13, 15, 17, 19, 21, or 23), but more preferably is a non-human homologue of a human SECX gene. For example, a mouse homologue of a human SECX gene can be used to construct a homologous recombination vector suitable for altering an endogenous SECX gene in the mouse genome. In one embodiment, the vector is designed such that, upon homologous recombination, the endogenous SECX gene is functionally disrupted (i.e., no longer encodes a functional protein; also referred to as a “knock out” vector).

Alternatively, the vector can be designed such that, upon homologous recombination, the endogenous SECX gene is mutated or otherwise altered but still encodes functional protein (e.g., the upstream regulatory region can be altered to thereby alter the expression of the endogenous SECX protein). In the homologous recombination vector, the altered portion of the SECX gene is flanked at its 5′- and 3′-termini by additional nucleic acid of the SECX gene to allow for homologous recombination to occur between the exogenous SECX gene carried by the vector and an endogenous SECX gene in an embryonic stem cell. The additional flanking SECX nucleic acid is of sufficient length for successful homologous recombination with the endogenous gene. Typically, several kilobases (Kb) of flanking DNA (both at the 5′-and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced SECX gene has homologously-recombined with the endogenous SECX gene are selected. See, e.g., Li, et al., 1992. Cell 69: 915.

The selected cells are then micro-injected into a blastocyst of an animal (e.g., a mouse) to form aggregation chimeras. See, e g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-human animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

### Pharmaceutical Compositions

The SECX nucleic acid molecules, SECX proteins, and anti-SECX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically-acceptable carrier. As used herein, “pharmaceutically-acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and other non-aqueous (i.e., lipophilic) vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a SECX protein or anti-SECX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

### Screening and Detection Methods

The nucleic acid molecules, proteins, protein homologues, and antibodies described herein can be used in one or more of the following methods: (A) screening assays; (B) detection assays (e.g., chromosomal mapping, cell and tissue typing, forensic biology), (C) predictive medicine (e.g., diagnostic assays, prognostic assays, monitoring clinical trials, and pharmacogenomics); and (D) methods of treatment (e.g., therapeutic and prophylactic).

The isolated nucleic acid molecules of the present invention can be used to express SECX protein (e.g., via a recombinant expression vector in a host cell in gene therapy applications), to detect SECX mRNA (e.g., in a biological sample) or a genetic lesion in an SECX gene, and to modulate SECX activity, as described further, below. In addition, the SECX proteins can be used to screen drugs or compounds that modulate the SECX protein activity or expression as well as to treat disorders characterized by insufficient or excessive production of SECX protein or production of SECX protein forms that have decreased or aberrant activity compared to SECX wild-type protein. In addition, the anti-SECX antibodies of the present invention can be used to detect and isolate SECX proteins and modulate SECX activity.

The invention further pertains to novel agents identified by the screening assays described herein and uses thereof for treatments as described, above.

### Screening Assays

The invention provides a method (also referred to herein as a “screening assay”) for identifying modulators, i.e., candidate or test compounds or agents (e.g., peptides, peptidomimetics, small molecules or other drugs) that bind to SECX proteins or have a stimulatory or inhibitory effect on, e.g., SECX protein expression or SECX protein activity. The invention also includes compounds identified in the screening assays described herein.

In one embodiment, the invention provides assays for screening candidate or test compounds which bind to or modulate the activity of the membrane-bound form of a SECX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead, one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of SECX protein, or a biologically-active portion thereof, on the cell surface is contacted with a test compound and the ability of the test compound to bind to a SECX protein determined. The cell, for example, can of mammalian origin or a yeast cell. Determining the ability of the test compound to bind to the SECX protein can be accomplished, for example, by coupling the test compound with a radioisotope or enzymatic label such that binding of the test compound to the SECX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of SECX protein, or a biologically-active portion thereof, on the cell surface with a known compound which binds SECX to form an assay mixture, contacting the assay mixture with a test compound, and determining the ability of the test compound to interact with a SECX protein, wherein determining the ability of the test compound to interact with a SECX protein comprises determining the ability of the test compound to preferentially bind to SECX protein or a biologically-active portion thereof as compared to the known compound.

In another embodiment, an assay is a cell-based assay comprising contacting a cell expressing a membrane-bound form of SECX protein, or a biologically-active portion thereof, on the cell surface with a test compound and determining the ability of the test compound to modulate (e.g., stimulate or inhibit) the activity of the SECX protein or biologically-active portion thereof. Determining the ability of the test compound to modulate the activity of SECX or a biologically-active portion thereof can be accomplished, for example, by determining the ability of the SECX protein to bind to or interact with a SECX target molecule. As used herein, a “target molecule” is a molecule with which a SECX protein binds or interacts in nature, for example, a molecule on the surface of a cell which expresses a SECX interacting protein, a molecule on the surface of a second cell, a molecule in the extracellular milieu, a molecule associated with the internal surface of a cell membrane or a cytoplasmic molecule. An SECX target molecule can be a non-SECX molecule or a SECX protein or polypeptide of the invention. In one embodiment, a SECX target molecule is a component of a signal transduction pathway that facilitates transduction of an extracellular signal (e.g. a signal generated by binding of a compound to a membrane-bound SECX molecule) through the cell membrane and into the cell. The target, for example, can be a second intercellular protein that has catalytic activity or a protein that facilitates the association of downstream signaling molecules with SECX.

Determining the ability of the SECX protein to bind to or interact with a SECX target molecule can be accomplished by one of the methods described above for determining direct binding. In one embodiment, determining the ability of the SECX protein to bind to or interact with a SECX target molecule can be accomplished by determining the activity of the target molecule. For example, the activity of the target molecule can be determined by detecting induction of a cellular second messenger of the target (i.e. intracellular Ca²⁺diacylglycerol, IP₃, etc.), detecting catalytic/enzymatic activity of the target an appropriate substrate, detecting the induction of a reporter gene (comprising a SECX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a SECX protein or biologically-active portion thereof with a test compound and determining the ability of the test compound to bind to the SECX protein or biologically-active portion thereof. Binding of the test compound to the SECX protein can be determined either directly or indirectly as described above. In one such embodiment, the assay comprises contacting the SECX protein or biologically-active portion thereof with a known compound which binds SECX to form an assay mixture, contacting the assay mixture with a test compound, and determining the ability of the test compound to interact with a SECX protein. wherein determining the ability of the test compound to interact with a SECX protein comprises determining the ability of the test compound to preferentially bind to SECX or biologically-active portion thereof as compared to the known compound.

In still another embodiment, an assay is a cell-free assay comprising contacting SECX protein or biologically-active portion thereof with a test compound and determining the ability of the test compound to modulate (e.g. stimulate or inhibit) the activity of the SECX protein or biologically-active portion thereof. Determining the ability of the test compound to modulate the activity of SECX can be accomplished, for example, by determining the ability of the SECX protein to bind to a SECX target molecule by one of the methods described above for determining direct binding. In an alternative embodiment, determining the ability of the test compound to modulate the activity of SECX protein can be accomplished by determining the ability of the SECX protein further modulate a SECX target molecule. For example, the catalytic/enzymatic activity of the target molecule on an appropriate substrate can be determined as described, above.

In yet another embodiment, the cell-free assay comprises contacting the SECX protein or biologically-active portion thereof with a known compound which binds SECX protein to form an assay mixture, contacting the assay mixture with a test compound, and determining the ability of the test compound to interact with a SECX protein, wherein determining the ability of the test compound to interact with a SECX protein comprises determining the ability of the SECX protein to preferentially bind to or modulate the activity of a SECX target molecule.

The cell-free assays of the invention are amenable to use of both the soluble form or the membrane-bound form of SECX protein. In the case of cell-free assays comprising the membrane-bound form of SECX protein, it may be desirable to utilize a solubilizing agent such that the membrane-bound form of SECX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl--N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either SECX protein or its target molecule to facilitate separation of complexed from non-complexed forms of one or both of the proteins, as well as to accommodate automation of the assay. Binding of a test compound to SECX protein, or interaction of SECX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-SECX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or SECX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, above. Alternatively, the complexes can be dissociated from the matrix, and the level of SECX protein binding or activity determined using standard techniques.

Other techniques for immobilizing proteins on matrices can also be used in the screening assays of the invention. For example, either the SECX protein or its target molecule can be immobilized utilizing conjugation of biotin and streptavidin. Biotinylated SECX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with SECX protein or target molecules, but which do not interfere with binding of the SECX protein to its target molecule, can be derivatized to the wells of the plate, and unbound target or SECX protein trapped in the wells by antibody conjugation. Methods for detecting such complexes, in addition to those described above for the GST-immobilized complexes, include immunodetection of complexes using antibodies reactive with the SECX protein or target molecule, as well as enzyme-linked assays that rely on detecting an enzymatic activity associated with the SECX protein or target molecule.

In another embodiment, modulators of SECX protein expression are identified in a method wherein a cell is contacted with a candidate compound and the expression of SECX mRNA or protein in the cell is determined. The level of expression of SECX mRNA or protein in the presence of the candidate compound is compared to the level of expression of SECX mRNA or protein in the absence of the candidate compound. The candidate compound can then be identified as a modulator of SECX mRNA or protein expression based upon this comparison. For example, when expression of SECX mRNA or protein is greater (i.e., statistically significantly greater) in the presence of the candidate compound than in its absence, the candidate compound is identified as a stimulator of SECX mRNA or protein expression. Alternatively, when expression of SECX mRNA or protein is less (statistically significantly less) in the presence of the candidate compound than in its absence, the candidate compound is identified as an inhibitor of SECX mRNA or protein expression. The level of SECX mRNA or protein expression in the cells can be determined by methods described herein for detecting SECX mRNA or protein.

In yet another aspect of the invention, the SECX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with SECX (“SECX-binding proteins” or “SECX-bp”) and modulate SECX activity. Such SECX-binding proteins are also likely to be involved in the propagation of signals by the SECX proteins as, for example, upstream or downstream elements of the SECX pathway.

The two-hybrid system is based on the modular nature of most transcription factors, which consist of separable DNA-binding and activation domains. Briefly, the assay utilizes two different DNA constructs. In one construct, the gene that codes for SECX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a SECX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with SECX.

The invention further pertains to novel agents identified by the aforementioned screening assays and uses thereof for treatments as described herein.

### Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

### Chromosome Mapping

Once the sequence (or a portion of the sequence) of a gene has been isolated, this sequence can be used to map the location of the gene on a chromosome. This process is called chromosome mapping. Accordingly, portions or fragments a SECX sequence, e.g. a portion or fragment of one or more of SEQ ID NO:1, 3, 5, 7, 9, 11, 13, 15, 17, 19, 21, or 23, or fragments or derivatives thereof, can be used to map the location of the SECX genes, respectively, on a chromosome. The mapping of the SECX sequences to chromosomes is an important first step in correlating these sequences with genes associated with disease.

Briefly, SECX genes can be mapped to chromosomes by preparing PCR primers (preferably 15-25 bp in length) from the SECX sequences. Computer analysis of the SECX sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the SECX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media. panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the SECX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to non-coding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Additionally, differences in the DNA sequences between individuals affected and unaffected with a disease associated with the SECX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

### Tissue Typing

The SECX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” as described in U.S. Pat. No. 5,272,057).

Furthermore, the sequences of the invention can be used to provide an alternative technique that determines the actual base-by-base DNA sequence of selected portions of an individual's genome. Thus, the SECX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The SECX sequences of the invention uniquely represent portions of the human genome. Allelic variation occurs to some degree in the coding regions of these sequences, and to a greater degree in the non-coding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the non-coding regions, fewer sequences are necessary to differentiate individuals. The non-coding sequences can comfortably provide positive individual identification with a panel of perhaps 10 to 1,000 primers that each yield a non-coding amplified sequence of 100 bases. If predicted SECX coding sequences, such as those in SEQ ID NOs: 1, 3, 5, 7, 9, 11, 13, 15, 17, 19, 21, or 23 are used, a more appropriate number of primers for positive individual identification would be 500-2,000.

### Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining SECX protein and/or nucleic acid expression as well as SECX activity, in the context of a biological sample (e.g., blood, serum, cells, tissue) to thereby determine whether an individual is afflicted with a disease or disorder, or is at risk of developing a disorder, associated with aberrant SECX expression or activity. The invention also provides for prognostic (or predictive) assays for determining whether an individual is at risk of developing a disorder associated with SECX protein, nucleic acid expression or activity. For example, mutations in a SECX gene can be assayed in a biological sample. Such assays can be used for prognostic or predictive purpose to thereby prophylactically treat an individual prior to the onset of a disorder characterized by or associated with SECX protein, nucleic acid expression or activity.

Another aspect of the invention provides methods for determining SECX protein, nucleic acid expression or SECX activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of SECX in clinical trials.

### Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining SECX protein and/or nucleic acid expression as well as SECX activity, in the context of a biological sample (e.g., blood, serum, cells, tissue) to thereby determine whether an individual is afflicted with a disease or disorder, or is at risk of developing a disorder, associated with aberrant SECX expression or activity. The invention also provides for prognostic (or predictive) assays for determining whether an individual is at risk of developing a disorder associated with SECX protein, nucleic acid expression or activity. For example, mutations in a SECX gene can be assayed in a biological sample. Such assays can be used for prognostic or predictive purpose to thereby prophylactically treat an individual prior to the onset of a disorder characterized by or associated with SECX protein, nucleic acid expression, or biological activity.

Another aspect of the invention provides methods for determining SECX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of SECX in clinical trials. These and other agents are described in further detail in the following sections.

### Diagnostic Assays

An exemplary method for detecting the presence or absence of SECX in a biological sample involves obtaining a biological sample from a test subject and contacting the biological sample with a compound or an agent capable of detecting SECX protein or nucleic acid (e.g., mRNA, genomic DNA) that encodes SECX protein such that the presence of SECX is detected in the biological sample. An agent for detecting SECX mRNA or genomic DNA is a labeled nucleic acid probe capable of hybridizing to SECX mRNA or genomic DNA. The nucleic acid probe can be, for example, a full-length SECX nucleic acid, or a portion thereof, such as an oligonucleotide of at least 15, 30, 50, 100, 250 or 500 nucleotides in length and sufficient to specifically hybridize under stringent conditions to SECX mRNA or genomic DNA. Other suitable probes for use in the diagnostic assays of the invention are described herein.

An agent for detecting SECX protein is an antibody capable of binding to SECX protein, preferably an antibody with a detectable label. Antibodies can be polyclonal, or more preferably, monoclonal. An intact antibody, or a fragment thereof (e.g., Fab or F(ab)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect SECX mRNA, protein, or genomic DNA in a biological sample in vitro as well as in vivo. For example, in vitro techniques for detection of SECX mRNA include Northern hybridizations and in situ hybridizations. In vitro techniques for detection of SECX protein include enzyme linked immunosorbent assays (ELISAs), Western blots, immunoprecipitations, and immunofluorescence. In vitro techniques for detection of SECX genomic DNA include Southern hybridizations. Furthermore, in vivo techniques for detection of SECX protein include introducing into a subject a labeled anti-SECX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting SECX protein, mRNA, or genomic DNA, such that the presence of SECX protein, mRNA or genomic DNA is detected in the biological sample, and comparing the presence of SECX protein, mRNA or genomic DNA in the control sample with the presence of SECX protein, mRNA or genomic DNA in the test sample.

The invention also encompasses kits for detecting the presence of SECX in a biological sample. For example, the kit can comprise: a labeled compound or agent capable of detecting SECX protein or mRNA in a biological sample; means for determining the amount of SECX in the sample; and means for comparing the amount of SECX in the sample with a standard. The compound or agent can be packaged in a suitable container. The kit can further comprise instructions for using the kit to detect SECX protein or nucleic acid.

### Prognostic Assays

The diagnostic methods described herein can furthermore be utilized to identify subjects having or at risk of developing a disease or disorder associated with aberrant SECX expression or activity. For example, the assays described herein, such as the preceding diagnostic assays or the following assays, can be utilized to identify a subject having or at risk of developing a disorder associated with SECX protein, nucleic acid expression or activity. Alternatively, the prognostic assays can be utilized to identify a subject having or at risk for developing a disease or disorder. Thus, the invention provides a method for identifying a disease or disorder associated with aberrant SECX expression or activity in which a test sample is obtained from a subject and SECX protein or nucleic acid (e.g., mRNA, genomic DNA) is detected, wherein the presence of SECX protein or nucleic acid is diagnostic for a subject having or at risk of developing a disease or disorder associated with aberrant SECX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant SECX expression or activity. For example, such methods can be used to determine whether a subject can be effectively treated with an agent for a disorder. Thus, the invention provides methods for determining whether a subject can be effectively treated with an agent for a disorder associated with aberrant SECX expression or activity in which a test sample is obtained and SECX protein or nucleic acid is detected (e.g., wherein the presence of SECX protein or nucleic acid is diagnostic for a subject that can be administered the agent to treat a disorder associated with aberrant SECX expression or activity).

The methods of the invention can also be used to detect genetic lesions in a SECX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a SECX-protein, or the mis-expression of the SECX gene. For example, such genetic lesions can be detected by ascertaining the existence of at least one of: (i) a deletion of one or more nucleotides from a SECX gene; (ii) an addition of one or more nucleotides to a SECX gene; (iii) a substitution of one or more nucleotides of a SECX gene, (iv) a chromosomal rearrangement of a SECX gene; (v) an alteration in the level of a messenger RNA transcript of a SECX gene, (vi) aberrant modification of a SECX gene, such as of the methylation pattern of the genomic DNA, (vii) the presence of a non-wild-type splicing pattern of a messenger RNA transcript of a SECX gene, (viii) a non-wild-type level of a SECX protein, (ix) allelic loss of a SECX gene, and (x) inappropriate post-translational modification of a SECX protein. As described herein, there are a large number of assay techniques known in the art which can be used for detecting lesions in a SECX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the SECX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a SECX gene under conditions such that hybridization and amplification of the SECX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qu Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a SECX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in SECX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in SECX can be identified in two dimensional arrays containing light-generated DNA probes as described in Cronin, et al., above. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the SECX gene and detect mutations by comparing the sequence of the sample SECX with the corresponding wild-type (control) sequence. Examples of sequencing reactions include those based on techniques developed by Maxim and Gilbert, 1977. Proc. Natl. Acad. Sci. USA 74: 560 or Sanger. 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the SECX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type SECX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with SI nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in SECX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a SECX sequence, e.g., a wild-type SECX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in SECX genes. For example, single-strand conformation polymorphism (SSP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control SECX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tebtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a SECX gene.

Furthermore, any cell type or tissue, preferably peripheral blood leukocytes, in which SECX is expressed may be utilized in the prognostic assays described herein. However, any biological sample containing nucleated cells may be used, including, for example, buccal mucosal cells.

### Pharmacogenomics

Agents, or modulators that have a stimulatory or inhibitory effect on SECX activity (e.g., SECX gene expression), as identified by a screening assay described herein can be administered to individuals to treat (prophylactically or therapeutically) disorders (e.g., cancer or immune disorders associated with aberrant SECX activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of SECX protein, expression of SECX nucleic acid, or mutation content of SECX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C 19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of SECX protein, expression of SECX nucleic acid, or mutation content of SECX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a SECX modulator, such as a modulator identified by one of the exemplary screening assays described herein.

### Monitoring of Effects During Clinical Trials

Monitoring the influence of agents (e.g., drugs, compounds) on the expression or activity of SECX (e.g., the ability to modulate aberrant cell proliferation and/or differentiation) can be applied not only in basic drug screening, but also in clinical trials. For example, the effectiveness of an agent determined by a screening assay as described herein to increase SECX gene expression, protein levels, or upregulate SECX activity, can be monitored in clinical trails of subjects exhibiting decreased SECX gene expression, protein levels, or down-regulated SECX activity. Alternatively, the effectiveness of an agent determined by a screening assay to decrease SECX gene expression, protein levels, or down-regulate SECX activity, can be monitored in clinical trails of subjects exhibiting increased SECX gene expression, protein levels, or up-regulated SECX activity. In such clinical trials, the expression or activity of SECX and, preferably, other genes that have been implicated in, for example, a cellular proliferation or immune disorder can be used as a “read out” or markers of the immune responsiveness of a particular cell.

By way of example, and not of limitation, genes including SECX genes, that are modulated in cells by treatment with an agent (e.g., compound, drug or small molecule) that modulates SECX activity (e.g., identified in a screening assay as described herein) can be identified. Thus, to study the effect of agents on cellular proliferation disorders, for example, in a clinical trial, cells can be isolated and RNA prepared and analyzed for the levels of expression of SECX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of SECX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a SECX protein, mRNA, or genomic DNA in the pre-administration sample; (iii) obtaining one or more post-administration samples from the subject; (iv) detecting the level of expression or activity of the SECX protein, mRNA, or genomic DNA in the post-administration samples; (v) comparing the level of expression or activity of the SECX protein, mRNA, or genomic DNA in the pre-administration sample with the SECX protein, mRNA, or genomic DNA in the post administration sample or samples; and (vi) altering the administration of the agent to the subject accordingly. For example, increased administration of the agent may be desirable to increase the expression or activity of SECX to higher levels than detected, i.e., to increase the effectiveness of the agent. Alternatively, decreased administration of the agent may be desirable to decrease expression or activity of SECX to lower levels than detected, i.e., to decrease the effectiveness of the agent.

### Methods of Treatment

The invention provides for both prophylactic and therapeutic methods of treating a subject at risk of (or susceptible to) a disorder or having a disorder associated with aberrant SECX expression or activity. These methods of treatment will be discussed more fully, below.

### Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e, are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof, or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

### Prophylactic Methods

In one aspect, the invention provides a method for preventing, in a subject, a disease or condition associated with an aberrant SECX expression or activity, by administering to the subject an agent that modulates SECX expression or at least one SECX activity. Subjects at risk for a disease that is caused or contributed to by aberrant SECX expression or activity can be identified by, for example, any or a combination of diagnostic or prognostic assays as described herein. Administration of a prophylactic agent can occur prior to the manifestation of symptoms characteristic of the SECX aberrancy, such that a disease or disorder is prevented or, alternatively, delayed in its progression. Depending upon the type of SECX aberrancy, for example, a SECX agonist or SECX antagonist agent can be used for treating the subject. The appropriate agent can be determined based on screening assays described herein.

### Therapeutic Methods

Another aspect of the invention pertains to methods of modulating SECX expression or activity for therapeutic purposes. The modulatory method of the invention involves contacting a cell with an agent that modulates one or more of the activities of SECX protein activity associated with the cell. An agent that modulates SECX protein activity can be an agent as described herein, such as a nucleic acid or a protein, a naturally-occurring cognate ligand of a SECX protein, a peptide, a SECX peptidomimetic, or other small molecule. In one embodiment, the agent stimulates one or more SECX protein activity. Examples of such stimulatory agents include active SECX protein and a nucleic acid molecule encoding SECX that has been introduced into the cell. In another embodiment, the agent inhibits one or more SECX protein activity. Examples of such inhibitory agents include antisense SECX nucleic acid molecules and anti-SECX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a SECX protein or nucleic acid molecule. In one embodiment, the method involves administering an agent (e.g., an agent identified by a screening assay described herein), or combination of agents that modulates (e.g., up-regulates or down-regulates) SECX expression or activity. In another embodiment, the method involves administering a SECX protein or nucleic acid molecule as therapy to compensate for reduced or aberrant SECX expression or activity.

Stimulation of SECX activity is desirable in situations in which SECX is abnormally down-regulated and/or in which increased SECX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., pre-clampsia).

### Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

### Prophylactic and Therapeutic Uses of the Compositions of the Invention

The SECX nucleic acids and proteins of the invention may be useful in a variety of potential prophylactic and therapeutic applications. By way of a non-limiting example, a cDNA encoding the SECX protein of the invention may be useful in gene therapy, and the protein may be useful when administered to a subject in need thereof.

As set forth in TABLE 1, the disclosed SECI polypeptide (SEQ ID NO:2) is related to the Human Phosphatidylethanolamine-Binding Protein (PEBP); the disclosed SEC2 polypeptide (SEQ ID NO:4) is related to the Human Uroplakin III Protein; the diclsoed SEC3 polypeptide (SEQ ID NO:6) is related to the Human Cadherin-6 Protein (Kidney-Cadherin); the disclosed SEC4 polypeptide (SEQ ID NO:8) is related to the Human Cadherin-6 Protein (Kidney-Cadherin); the disclosed SEC5polypeptide (SEQ ID NO:10) is related to the Human Lymphocyte-Associated Receptor of Death 2; the disclosed SEC6 polypeptide (SEQ ID NO:12) is related to the Human Semaphorin Protein; the disclosed SEC7 polypeptide (SEQ ID NO:14) is related to the Human Semaphorin Protein; the disclosed SEC8 polypeptide (SEQ ID NO:16) is related to the Human Diazepam Binding Inhibitor (DBI) Protein; the disclosed SEC9 polypeptide (SEQ ID NO: 18) is related to the Aquifex aeolicus ATP Synthase A Chain Protein; the disclosed SEC10 polypeptide (SEQ ID NO:20) is related to the Human Lymphocyte-Associated Receptor of Death 2; the disclosed SEC 11 polypeptide (SEQ ID NO:22) is related to the Human Semaphorin Protein; and the disclosed SEC 12 polyeptide (SEQ ID NO:24) is related to the Human Diazepam Binding Inhibitor (DBI) Protein. The putative biological functions and any associated disorders of each of these proteins has been discussed, above.

Both the novel nucleic acids encoding the SECX proteins, and the SECX proteins of the invention, or fragments thereof, may also be useful in diagnostic applications, wherein the presence or amount of the nucleic acid or the protein are to be assessed. These materials are further useful in the generation of antibodies that immunospecifically-bind to the novel substances of the invention for use in therapeutic or diagnostic methods.

The invention will be further illustrated in the following non-limiting examples.

### EXAMPLE 1

### Chromosomal localization of a SEC8 Nucleic Acid by Radiation Hybrid Mapping

Radiation hybrid mapping using human chromosome markers was performed in the identification, development, and characterization of many of the SECX clones of the present invention. The procedure used to obtain these results is a modification of the method originally described in Steen, et al., 1999. A High-Density Integrated Genetic Linkage and Radiation Hybrid Map of the Laboratory Rat, Genome Res. 9: API-AP8 (Published Online on May 21, 1999).

A panel of 93 cell clones containing randomized radiation-induced human chromosomal fragments was screened in 96 well plates using PCR primers designed to identify the clones of interest. For example, using this method, a nucleic acid sequence encoding the SEC8 protein was found on chromosome 11 at a map distance of -0.7 cR from WI-4920 and -3.90 cR from WI-1421.

### EXAMPLE 2

### Molecular Cloning of an SEC1 Nucleic Acid

Oligonucleotide primers were designed to amplify using the polymerase chain reaciton (PCR) a DNA segment coding for the mature form of an SEC1 protein (Identification No. 3445452; SEQ ID NO:2) from amino acid residues 23 to 227. The forward primer (SEQ ID NO:25) includes an in-frame BglII restriction site and the reverse primer (SEQ ID NO:26) contains an in-frame SalI restriction site. The sequences of the primers are:

SEC1 MatF: AGATCT GAC GAG GAT GAG AAC AGC CCG (SEQ ID NO:25)

SEC1 Rev: CTCGTC GTCGAC GCA GGC AGC TAT CTC CGC CTG GTT TTT GTG (SEQ ID NO:26)

Each PCR reaction included 5 ng human testis cDNA template; 1 μM of each of the SEC1 MatF and SEC1 Rev primers; 5 μM dNTPs (Clontech Laboratories; Palo Alto, Calif.); and 1 μl of 50× Advantage-HF 2 polymerase (Clontech Laboratories; Palo Alto, Calif.) in a 50 μl total reaction volume. The following PCR reaction conditions were used:

(a) 96° C. 3 minutes
(b) 96° C. 30 seconds, denaturation
(c) 60° C. 30 seconds, primer annealing.
(d) 72° C. 1 minute, extension.
Repeat steps (b)-(d) a total of 35-times.
(e) 72° C. 5 minutes, final extension.

A single 650 bp amplified product was detected by agarose gel electrophoresis. The product was isolated and ligated into the pCR2. 1 cloning vector (Invitrogen, Carlsbad CA) The DNA sequence of the cloned insert was determined and found to include an ORF coding for a 205 amino acid residue polypeptide. The construct was designated 3445452-pCR2.1-S262-1C, and the nucleotide sequence of the insert is shown in FIG. 1 (SEQ ID NO:1).

In FIG. 1, the underlined nucleotides at the 5′- and 3′-termini originate from the cloning site within the vector. Thus, these sequences do not represent SEC1 nucleotides.

The amino acid sequence of the encoded polypeptide is also shown in FIG. 1 (SEQ ID NO:2). The underlined residues in FIG. 1 at the amino- and carboxyl-termini originate from the cloning site within the vector, and do not represent SEC1 amino acid residues.

### Example 3

### Construction of Mammalian Expression Vector Pcep4/Sec

An expression vector, named Pcep4/Sec, which allows heterologous protein expression and secretion by fusing any protein to the Igk chain signal peptide was constructed.

To construct pcep4/Sec, two oligonucleotide primers pSec-V5-His Forward (SEQ ID NO:27) and pSec-V5-His Reverse (SEQ ID NO:28) were produced to amplify a fragment from the pcDNA3.1-V5His expression vector (Invitrogen; Carlsbad, Calif.) that includes V5 and His6. The sequences of the two primers are shown below:

pSec-V5-His Forward: CTCGTCCTCGAGGGTAAGCCTATCCCTAAC (SEQ ID NO:27)

pSec-V5-His Reverse: CTCGTCGGGCCCCTGATCAGCGGGTTTAAAC (SEQ ID NO:28)

The PCR amplified product was then digested with XhoI and ApaI and ligated into the XhoI/ApaI-digested pSecTag2 B vector possessing an IgK leader sequence (Invitrogen; Carlsbad, Calif.). The structure of the resulting vector (designated pSecV5His), including an in-frame IgK leader sequence and V5-His6, was verified by DNA sequence analysis.

The vector pSecV5His was digested with PmeI and NheI to provide a fragment retaining the aforementioned sequences in the correct frame. The PmeI-NheI fragment was then ligated into the BamHI/Klenow- and NheI-treated vector pCEP4 (Invitrogen; Carlsbad, Calif.). The resulting vector was designated pCEP4/Sec, and included an in-frame IgK leader sequence, a site for insertion of a clone of interest, V5 and His6 sequences under the control of the PCMV and/or the PT7 promoter.

Detection and purification of the expressed protein is aided by the presence of the V5 epitope tag and 6X His tag at the carboxyl-terminus (Invitrogen; Carlsbad, Calif.).

### EXAMPLE 4

### Expression of the Mature Form of a SEC1 Polypeptide (3445452) in HEK 293 Cells

A BamHI-SalI fragment containing a SEC1 sequence (SEQ ID NO:1) (Identification No.3445452) was isolated from the pCR2.1-cg3445452-S262-1C construct and then subcloned into the pCEP4/Sec vector (Example 3) to generate an expression vector construct designated pCEP4/Sec-3445452.

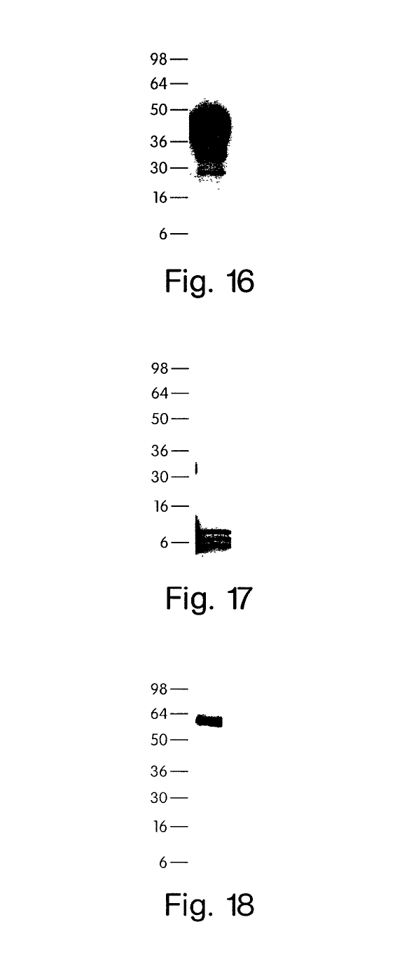

The pCEP4/Sec-3445452 vector was then transfected into human embryonic kidney 293 cells (ATCC No. CRL- 1573; Manassas, Va.) using the LipofectaminePlusa™ Reagent and following the manufacturer's instructions (GibcolBRL/Life Technologies; Rockville, Md.). The cell pellet and supernatant were harvested 72 hours after transfection and examined for h3445452 expression. FIG. 16 illustrates that Western blotting (reducing conditions) with an anti-V5 antibody shows 3445452 was expressed as a secreted protein with an apparent molecular weight of 40 kiloDaltons (kDa)

The mature protein is predicted to have a single N-glycosylation site at Asnl47. It is also believed that the apparent molecular weight of the protein product and the diffuse appearance of the protein band following electrophoretic separation, may be due to glycosylation of the protein (including the presence of diverse carbohydrate chains on the glycoprotein).

### EXAMPLE 5

### Molecular Cloning of the Extracellular Domain of a SEC2 Polypeptide

The predicted open reading frame (ORF) of the SEC2 nucleic acid (SEQ ID NO:3) (Identification No. 4011999; SEQ ID NO:3) encodes a novel, Type I Transmembrane protein. Oligonucleotide primers were produced to allow PCR amplification of nucleotides encoding amino acid residues 1- 1 97. These nucleotides correspond to an extracellular domain of the disclosed SEC2 polypeptide. The forward primer, SEC2 F-Topo-Forward, (SEQ ID NO:29) includes an in frame BamHI restriction site followed by the triplet of bases ACC to form a consensus Kozak site, whereas the reverse primer, SEC2 F-Topo-Reverse, (SEQ ID NO:30) contains an in frame XhoI restriction site. The sequences of the two primers are shown below:

SEC2 F-Topo-Forward: GGATCC ACC ATG GTG CGA ACG CGG TGG CAG CCT CAC (SEQ ID NO:29)

SEC2 F-Topo-Reverse: CTCGAG ACA GCC GCT CCG TCG GCC AGG CCA TGT (SEQ ID NO:30)

Each PCR reaction included 5 ng mouse testis cDNA template; 1 μM of each of the SEC2 F-Topo-Forward and SEC2 F-Topo-Reverse primers; 5 μM dNTPs (Clontech Laboratories; Palo Alto, Calif.); and 1 μl of 50× Advantage-HF 2 polymerase (Clontech Laboratories; Palo Alto, Calif.) in a 50 μl total reaction volume. The following PCR reaction conditions were used:

| | | |
|---|---|---|
| (a) | 96° C. | 3 minutes |
| (b) | 96° C. | 30 seconds denaturation |
| (c) | 70° C. | 30 seconds, primer annealing. This temperature was |
| | | gradually decreased by 1° C./PCR cycle. |
| (d) | 72° C. | 1 minute extension. |

Repeat steps (b)-(d) a total of 10-times

| | | |
|---|---|---|
| (e) | 96° C. | 30 seconds, denaturation |
| (f) | 60° C. | 30 seconds, annealing |
| (g) | 72° C. | 1 minute, extension |

Repeat steps (e)-(g) a total of 25-times

| | | |
|---|---|---|
| (h) | 72° C. | 5 minutes, final extension. |

A single, amplified PCR product of approximately 570 bp was detected by agarose gel electrophoresis. The nucleic acid was then isolated and inserted into the pCDNA3. 1 -V5-TOPO vector (Invitrogen; Carlsbad, Calif.) by a topoisomerase I-mediated cloning method. The insert sequence was subsequently determined to be an open reading frame (ORF) encoding a polypeptide comprising 197 amino acid residues. The resulting construct was designated 4011999-pCDNA3.1-TOPO-S69-A. The nucleotide sequence of the insert nucleic acid was determined to be 100% identical to the corresponding portion of the sequence shown in FIG. 2 (SEQ ID NO:3).

### EXAMPLE 6

### Cloning of a Nucleic Acid Encoding an Extracellular Domain of the Mature Form of an SEC2 Polypeptide

Two oligonucleotide primers were designed to PCR amplify a sequence within a region of the disclosed SEC2 nucleic acid (SEQ ID NO:3) encoding an extracellular domain of the mature SEC2 polypeptide.

The forward primer, SEC2 C-Forward, (SEQ ID NO:31) included an in-frame BamHI restriction site, whereas the reverse primer, SEC2 SECR, (SEQ ID NO:32) included an in-frame XhoI restriction site. The sequences of the two are shown below:

SEC2 C-Forward: GACGTC GGATCC CTA GAC CTG ATT GCC TAC GTG CCG CAG (SEQ ID NO:31)

SEC2 SECR: CTCGTC CTCGAG ACA GCC GCT CCG TCG GCC AGG CCA TGT G (SEQ ID NO:32)

Each PCR reaction was comprised of: 1ng of cgm401ng of cgm4011999-pCDNA3.1-TOPO-S69-A template; 1 μM of each of the SEC2 C-Forward and SEC2 SECR primers; 5 μM dNTPs (Clontech Laboratories; Palo Alto, Calif.); and 1 μl of 50× Advantage-HF 2 polymerase (Clontech Laboratories; Palo Alto, Calif.) in a 50 μl total reaction volume. The following PCR reaction conditions were used:

(a) 96° C. 3 minutes
(b) 96° C. 30 seconds, denaturation
(c) 60° C. 30 seconds, primer annealing.
(d) 72° C. 1 minute, extension.
Repeat steps (b)-(d) a total of 15-times.
(e) 72° C. 5 minutes, final extension.

A single, amplified PCR product of approximately 480 bp was detected by agarose gel electrophoresis. The nucleic acid was then isolated, digested with BamHI and XhoI, and inserted into the pSecV5His vector. The resulting construct was designated 4011999-pSecV5His-S 151 -A. The insert sequence was subsequently determined to be an open reading frame (ORF) encoding a polypeptide comprising 170 amino acid residues that are 100% identical to the corresponding portion of the SEC2 amino acid sequence shown in FIG. 2 (SEQ ID NO:4).

### EXAMPLE 7

### Expression of a SEC2 Polypeptide in Human Embryonic Kidney 293 Cells

The BamHI-XhoI fragment containing a SEC2 nucleic acid sequence was isolated from the pSecV5His-cg4011999-S15 1-A construct (see, Example 6) and subcloned into the vector pCEP4/Sec (see, Example 3) to generate an expression vector construct designated, pCEP4/Sec-4011999. The pCEP4/Sec-4011999 construct was then transfected into 293 cells using the LipofectaminePlus Reagent and following the manufacturer's instructions (Gibco/BRL/Life Technologies; Rockville, Md.). The cell pellet and supernatant were harvested 72 hours after transfection and examined for SEC2 polypeptide expression by Western blotting (reducing conditions) with an anti-V5 antibody. FIG. 17 shows that the V5-detected SEC2 product appears as three discrete bands of apparent molecular weights of approximately 6-10 kDa, when expressed in, and secreted by 293 cells. It should be noted that these molecular weights are lower than the value expected and it is presumed that post-translational proteolysis occurs either extracellularly or intracellularly to yield the observed electrophoretic bands. Evidence for proteolysis has also been observed with in this system in other cases (data not shown).

### EXAMPLE 8

### Molecular Cloning of a Nucleic Acid Encoding a SEC10 Polypeptide

The predicted open reading frame (ORF) of a SEC10 nucleic acid (SEQ ID NO: 19) ((Identification No. 1795045.0.77) encodes a polypeptide comprising 464 amino acid residues. Oligonucleotide primers were produced to facilitate the PCR-mediated amplification of the sequence encoding amino acid residues 1 to 391 in the ORF. The forward primer, SEC10 Forward, (SEQ ID NO:33) included a CTCGTC clamp and a BglII restriction site, whereas the reverse primer, SEC 10 Reverse, (SEQ ID NO:33) included a CTCGTC clamp and an in-frame XhoI restriction site. The sequences of the two primers are shown below:

SEC10 Forward: CTCGTC AGATCT ATG AAG AAC CAG GTA TGC AGT AAG TGT G (SEQ ID NO:33)

SEC10 Reverse: CTCGTC CTCGAG GGC TCC AGT CAT AGA TGT TGG TGG TTT AAA (SEQ ID NO:34)

Each PCR reaction was comprised of: 5 ng human thalamus cDNA template; 1 μM of each of the SEC 10 Forward and SEC 10 Reverse primers; 5 μM dNTPs (Clontech Laboratories; Palo Alto, Calif.); and 1 μl of 50× Advantage-HF 2 polymerase (Clontech Laboratories; Palo Alto, Calif.) in a 50 μI total reaction volume. The following PCR reaction conditions were used:

| | | |
|---|---|---|
| (a) | 96° C. | 3 minutes |
| (b) | 96° C. | 30 seconds, denaturation. |
| (c) | 70° C. | 30 seconds, primer annealing. This temperature was |
| | | gradually decreased by 1° C./PCRcycle. |
| (d) | 72° C. | 3 minutes, extension. |

Repeat steps (b)-(d) a total of 10-times.

| | | |
|---|---|---|
| (e) | 96° C. | 30 seconds, denaturation. |
| (f) | 60° C. | 30 seconds, annealing. |
| (g) | 72° C. | 3 minutes, extension. |

Repeat steps (e)-(g) a total of 25-times.

| | | |
|---|---|---|
| (h) | 72° C. | 10 minutes, final extension. |

A single, amplified PCR product of approximately 1.2 Kbp was detected by agarose gel electrophoresis. The nucleic acid product was then isolated and ligated into the pCR2.1 vector (Invitrogen; Carlsbad, Calif.), and designated pCR2.1-cgl 1795045-S181-2. The construct was sequenced and verified as being 100% identical to the sequence of clone 1795045.0.77 coding for residues 1-391.

### EXAMPLE 9

### Expression of a SEC10 Polypeptide in Human Rmbryonic Kidney 293 Cells

The BamHI-SalI fragment containing the disclosed SEC10 nucleic acid sequence (SEQ ID NO: 19) (Identitifcation No. 1795045.0.77) sequence was isolated from the pCR2.-cg1795045-S 181-2 construct and subcloned into the pCEP4/Sec vector to generate an expression vector construct designated pCEP4/Sec-1795045. The pCEP4/Sec-1795045 construct was then transfected into HEK 293 cells using the LipofectaminePlus™ Reagent and following the manufacturer's instructions (Gibco/BRL/Life Technologies; Rockville, Md.). The cell pellet and supernatant were harvested 72 hours after transfection and examined for SEC 10 expression by Western blotting (reducing conditions) with an anti-V5 antibody. FIG. 18 shows that SEC 10 is expressed as a protein of an apparent molecular weight of approximately 63 kDa when expressed in and secreted by 293 cells.

The predicted molecular weight for the SEC10 protein is approximately is 46 kDa, thus it is possible that the higher observed molecular weight may be due to glycosylation of the protein. The program PROSITE predicts three N-glycosylation sites for the SEC 10 polypeptide (i.e., at AsnI 11, Asn238, and Asn393).

### EXAMPLE 10

### Expression Analysis of SECX Nucleic Acid Sequences

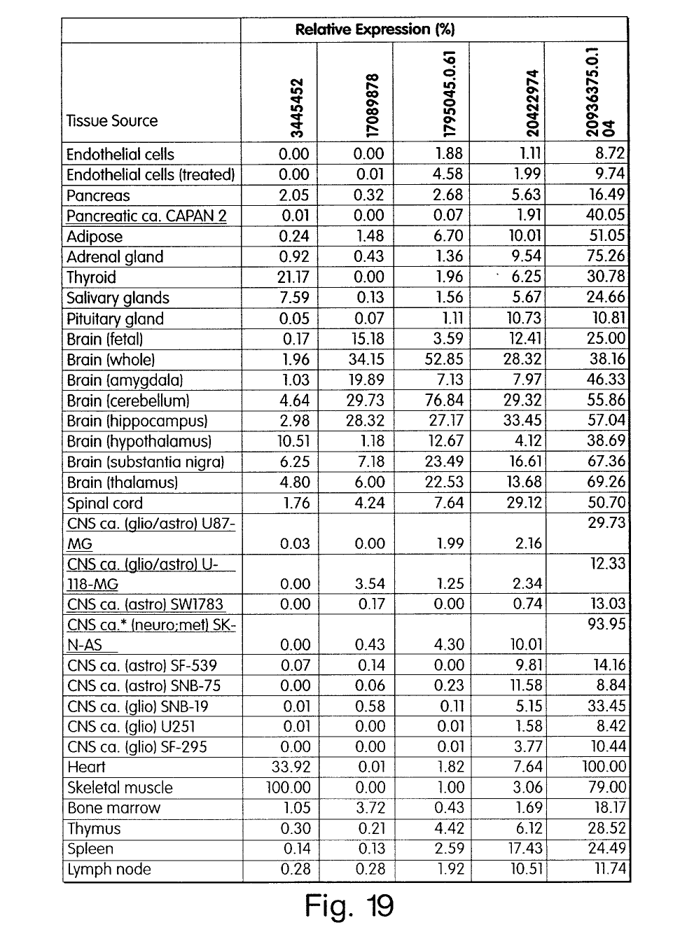

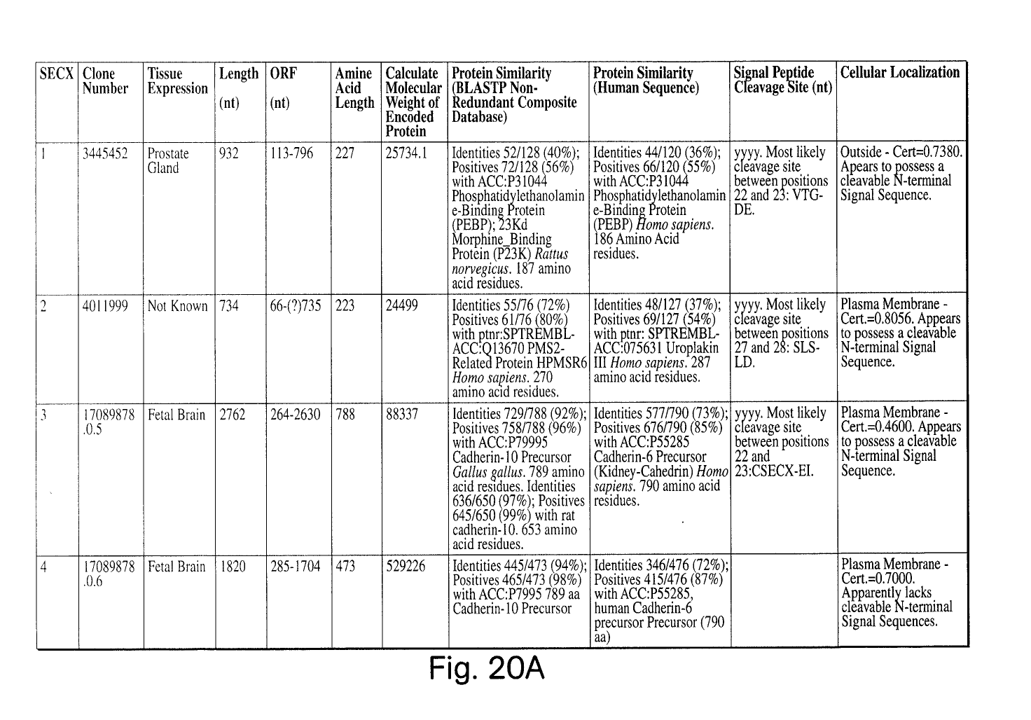

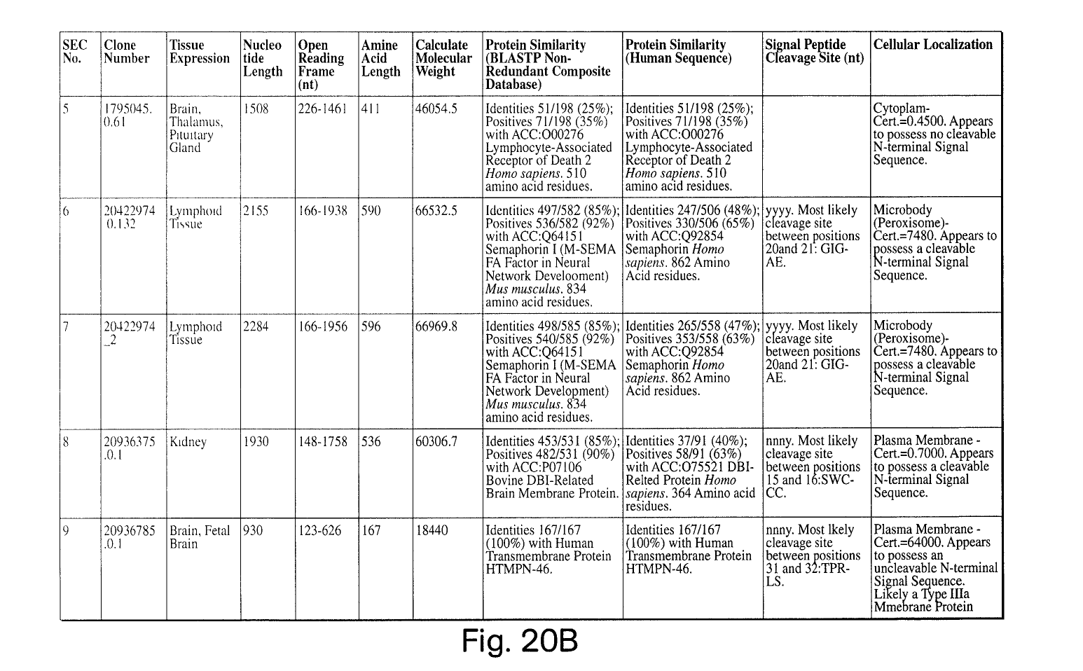

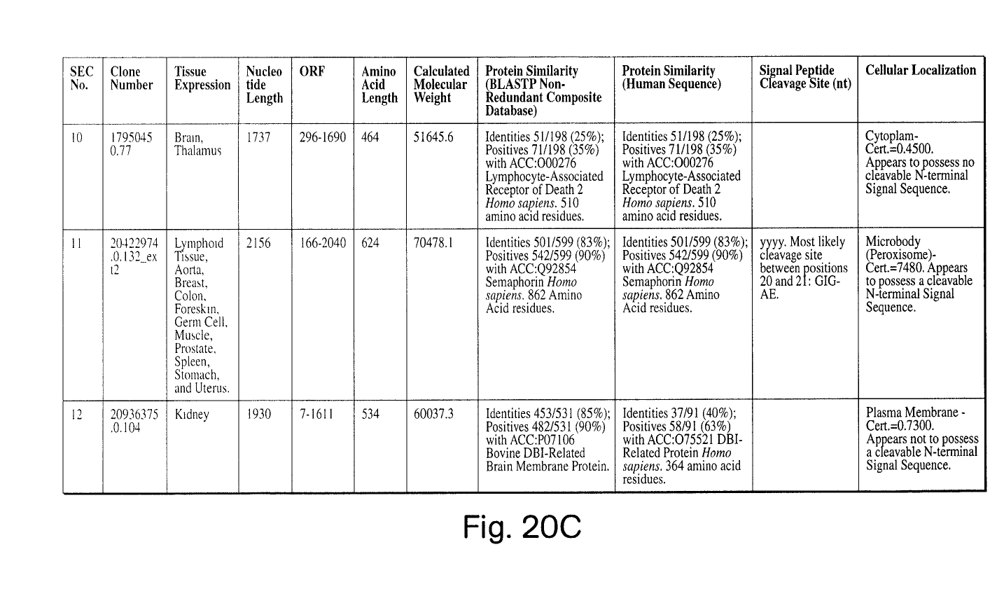

The quantitative expression of several SECX clones was assessed in 41 normal and 55 tumor samples by real-time quantitative PCR (TAQMAN® expression analaysis) performed on a Perkin-Elmer Biosystems ABI PRISM® 7700 Sequence Detection System. The results are shown In FIG. 19, the following abbreviations are used:

ca. =Carcinoma

* =Established from Metastasis

met =Metastasis

s cell var =Small Cell Variant

non-s =Non-Small

squam =Squamous

pl. eff =Pleural Effusion

glio =Glioma

astro =Astrocytoma

neuro =Neuroblastoma

Initially, 96 RNA samples were normalized to β-actin and GAPDH. RNA (˜50 ng total or ˜1 ng poly(A)⁺) was converted to cDNA using the TAQMAN® Reverse Transcription Reagents Kit (PE Biosystems; Foster City, Calif.; Catalog No. N808-0234) and random hexamers according to the manufacturer's protocol. Reactions were performed in a 20 μl total volume and incubated for 30 min. at 48° C. cDNA (5 μl of the reaction mixture) was then transferred to a separate plate for the TAQMANd® reaction using β-actin and GAPDH TAQMAN® Assay Reagents (PE Biosystems; Foster City, Calif.; Catalog Nos. 4310881E and 4310884E, respectively) and TAQMAN® universal PCR Master Mix (PE Biosystems; Foster City, Calif.; Catalog No. 4304447) according to the manufacturer's protocol. Reactions were performed in a total volume of 25 μl using the following parameters: 2 min. at 50° C.; 10 min. at 95° C.; and 15 sec. at 95° C./1 min. at 60° C (for a total of 40 cycles).

Results were recorded as CT values (i.e., the cycle at which a given sample crosses a threshold level of fluorescence) using a log scale, wherein the difference in RNA concentration between a given sample and the sample with the lowest CT value was represented as 2 to the power of delta CT. The percent relative expression was then obtained by taking the reciprocal of this RNA difference and multiplying by 100. The average CT values obtained for β-actin and GAPDH were used to normalize RNA samples. The RNA sample generating the highest CT value required no further diluting, while all other samples were diluted relative to this sample according to their β-actin /GAPDH average CT values.

Normalized RNA (5 μl) was converted to cDNA and analyzed via TAQMAN® using One-Step RT-PCR Master Mix Reagents (PE Biosystems; Foster City, Calif.; Catalog No. 4309169) and gene-specific primers according to the manufacturer's instructions. Probes and primers were designed for each assay according to Perkin Elmer Biosystem's Primer Express Software package (Version I for Apple Computer's Macintosh Power PC) using the sequence of the respective clones as input. Default settings were used for reaction conditions and the following parameters were established before selecting primers: (i) primer concentration =250 nM; (ii) primer melting temperature (Tm) range=58°-60° C.; (iii) primer optimal Tm=59° C.; (iv) maximum primer difference=2° C.; (v) probe does not have 5′-terminal G; (vi) probe Tm must be 10° C. greater than primer T_{m;} and (vii) amplicon size must be 75 bp to 100 bp. The probes and primers selected (see below) were synthesized by Synthegen (Houston, Tex.). Probes were double-purified by HPLC to remove uncoupled dye and evaluated by mass spectroscopy to verify coupling of reporter and quencher dyes to the 5′- and 3′-termini, respectively. The final concentrations in the reactions were: forward and reverse primers=900 nM each; and probe=200nM.

Normalized RNA from each tissue and each cell line was ten “SECXotted” in each well of a 96 well PCR plate (Perkin Elmer Biosystems). PCR reaction mixtures (including two probes (a SECX-specific probe and another gene-specific probe multiplexed with the SECX probe) were prepared using 1X TaqMan™ PCR Master Mix for the PE Biosystems 7700, which contained: 5 mM MgCl₂; dNTPs (dATP, dGTP, dCTP, and dUTP at 1:1:1:2 ratios); 0.25 U/ml AmpliTaq Gold™ (PE Biosystems; Foster City, Calif.); 0.4 U/μl RNase inhibitor; and 0.25 U/μl reverse transcriptase. Reverse transcription was then performed at 48° C. for 30 minutes followed by PCR-mediated amplification cycles as follows: 95° C. 10 minute; 40 cycles of 95° C. for 15 seconds; and 60° C. for 1 minute. The primer-probe sets employed in the expression analysis of each clone, and a summary of the results, are provided below:

### SEC1 (3445452)

Ag 36 (F): 5′-CAGGTGGAAACGGTTCAGAAA-3′ (SEQ ID NO:35)

Ag 36 (R): 5′-CATCTCTCTCCTTCCCAAGGAA-3′ (SEQ ID NO:36)

Ag 36 (P): FAM-5′-CTGTCCATTTTCCAAGAGCCTCGAGTTTTGT- 3′-TAMRA (SEQ ID NO:37)

SEC 1 is primarily expressed in normal tissues such as thyroid, hypothalamus, heart, skeletal muscle, lung, testis, and prostate.

### SEC3 (17089878.0.5) and SEC4 (17089878.0.6) (Generically Designated as 17089878 in FIG. 19)z

Ag 123 (F): 5′-CAGGCACACTGACCATTCGA-3′ (SEQ ID NO:38)

Ag 123(R): 5′-GAGCAGGGCTTCAGCACTG-3′ (SEQ ID NO:39)

Ag 123 (P): FAM-5′-TGCCTTGGCTGTCACAAGCACACA-3′-TAMRA (SEQ ID NO:40)

Transcripts homologus to the SEC3 and SECr probes were primarily detected in normal tissues including diverse classes of brain tissue, in liver and in lung large cell carcinoma.

### SEC5 (1795045.0.61)

Ag80(F): 5′-CAGAGGAAGGATCCAGTGAGTGT-3′ (SEQ ID NO:41)

Ag80(R): 5′-CATGGAGTATGGATCTGGAAATAGTC-3′ (SEQ ID NO:42)

Ag80(P): FAM-5′-CAGAGCGCCCTCCCTGTACCACAAA-3′-TAMRA (SEQ ID NO:43)

SEC5 was found to be expressed in most normal brain tissues, mammary gland, colon cancer HCT-116 cells, gastric cancer cells, lung small cell cancer tissues, lung non-small cell cancer, lung squamous cell cancer, and in prostate cancer metastases.

### SEC6 (20422974.0.132); SEC7(20422974.2), and SEC11 (20422974.0.132) (Generically Designated 20422974 in FIG. 19)

Ag 37(F): 5′-GGGAGTGGGCCTGACTTTCT-3′ (SEQ ID NO:44)

Ag 37(R): 5′-GCATGTGATGACCTCGGACA-3′ (SEQ ID NO:45)

Ag 37(P): FAM-5′-TTCAGGCATCTGCAACCTCCGTGG-3′-TAMRA (SEQ ID NO:46)

SEC6, SEC7, and SEC 11 are expressed in adipose tissue, diverse brain tissues and spinal cord, central nervous system (CNS) cancers, spleen, lymph node, colon cancer HCT-116 cells, fetal kidney, fetal lung, lung small cell, large cell, non-small cell and squamous carcinomas, mammary gland, breast cancer, ovary and ovarian cancer cells, placenta, prostate and prostate cancer bone metastasis cells, and melanoma tissues.

### SEC12 (20936375.0.104)

Ag 174(F): 5′-AGGACATAGGATGCAACACTTGAG-3′ (SEQ ID NO:47)

Ag 174(R): 5′-CCAGCGCTCCCCATCAC-3′ (SEQ ID NO:48)

Ag 174(P): TET-5′-ACCTGCCGGCCCTTGGTTCCT-3z′-TAMRA (SEQ ID NO:49)

The results show that SEC12 is widely expressed at high levels in most normal and cancerous tissues.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A substantially purified polypeptide comprising an amino acid sequence selected from any one of the following:
(a) a polypeptide of SEQ ID NO: 12;
(b) a polypeptide having one or more conservative amino acid substitutions to the polypeptide of SEQ ID NO: 12; or
(c) a mutant or variant of the polypeptide of SEQ ID NO: 12.

2. A vector which encodes for the polypeptide of claim 1.

3. A cell comprising the vector of claim 2.

4. The cell of claim 3, wherein said cell is a prokaryotic or eukaryotic cell.

5. A process of producing a polypeptide of SEQ ID NO: 12, the process comprising:
(a) providing the cell of claim 4;
(b) culturing said cell under conditions sufficient to express the SEQ ID NO: 12 polypeptide; and
(c) recovering said SEQ ID NO: 12 polypeptide, thereby producing said SEQ ID NO: 12 polypeptide.

6. A method of diagnosing a pathological condition associated with aberrant SEQ ID NO: 12 polypeptide expression or activity in a subject, the method comprising:
(a) providing a protein sample from said subject;
(b) providing a control protein sample;
(c) measuring the amount of SEQ ID NO: 12 polypeptide in said subject sample; and
(d) comparing the amount of SEQ ID NO: 12 polypeptide in said subject protein sample to the amount of SEQ ID NO: 12 polypeptide in said control protein sample,
wherein a difference in the amount of SEQ ID NO: 12 polypeptide in said subject protein sample relative to the amount of SEQ ID NO: 12 polypeptide in said control protein sample indicates the subject has said pathological condition.

7. The method of claim 6, wherein said SEQ ID NO: 12 polypeptide is detected using an antibody.

8. The method of claim 6, wherein said pathological condition is cancer.

9. A method for treating, preventing or delaying a pathological condition
associated with aberrant SEQ ID NO: 12 expression or activity in a subject, the method comprising administering to a subject in which said treatment, prevention or delay is desired the polypeptide of claim 1 in amount sufficient to treat, prevent or delay said pathological condition in said subject.

10. A method for identifying a compound that binds the polypeptide of claim 1, the method comprising:
(a) contacting SEQ ID NO: 12 protein with a compound; and
(b) determining whether said compound binds SEQ ID NO: 12 protein.

11. The method of claim 10, wherein binding of said compound to SEQ ID NO: 12 is determined by a protein assay.

12. A compound identified by the method of claim 11.

13. A method for identifying a compound that modulates the activity of a SEQ ID NO: 12 protein, the method comprising:
(a) contacting SEQ ID NO: 12 protein with a compound; and
(b) determining whether SEQ ID NO: 12 protein activity has been altered.

14. A compound identified by the method of claim 13.

15. A pharmaceutical composition comprising the polypeptide of claim 1 and a pharmaceutically-acceptable carrier.
